(19) United States
(12) Patent Application Publication
(10) Pub. No.: US 2002/0150626 A1
(43) Pub. Date: Oct. 17, 2002

(54) Lipid-protein-sugar particles for delivery of nucleic acids

(76) Inventors: Daniel S. Kohane, Newton, MA (US); Daniel G. Anderson, Framingham, MA (US); Robert S. Langer, Newton, MA (US)

Correspondence Address: Choate, Hall & Stewart, Exchange Place, 53 State Street, Boston, MA, 02109, US

(21) Appl. No.: 09/981,460

(22) Filed: Oct. 16, 2001

Related U.S. Application Data
(60) Provisional application No. 60/240,698, filed on Oct. 16, 2000.

(51) Int. Cl. A61K 48/00; A61K 9/16; A61K 9/50

(52) U.S. Cl. 424/493; 514/44

(57) ABSTRACT

Lipid-protein-sugar particles (LPSPs) are provided as a vehicle for the delivery of nucleic acids. Any polynucleotide (e.g., DNA, RNA) may be encapsulated in a lipid-protein-sugar matrix to form microparticles. Preferably the diameter of the LPSP ranges from 50 nm to 10 micrometers. The particles may be prepared using any known lipid (e.g., DPPC), protein (e.g., albumin), or sugar (e.g., lactose). Methods of preparing the particles and administering the particles for gene therapy are also provided. Preferably the methods of preparing the LPSPs do not significantly damage the polynucleotide to be delivered.

### Related Applications

The present application claims priority to co-pending provisional applications, U.S. Ser. No. 60/240,698, filed Oct. 16, 2000, which is incorporated herein by reference.

### BACKGROUND OF THE INVENTION

The delivery of a drug to a patient with controlled-release of the active ingredient has been an active area of research for decades and has been fueled by the many recent developments in polymer science and the need to deliver more labile pharmaceutical agents such as nucleic acids, proteins, and peptides. Biodegradable particles have been developed as sustained release vehicles used in the administration of small molecule drugs as well as protein and peptide drugs and nucleic acids (Langer Science 249:1527-1533, 1990; Mulligan Science 260:926-932, 1993; Eldridge Mol. Immunol. 28:287-294, 1991; each of which is incorporated herein by reference). The agents to be delivered are typically encapsulated in a polymer matrix which is biodegradable and biocompatible. As the polymer is degraded and/or as the agent diffuses out of the polymer, the agent is released into the body. Typical polymers used in preparing these particles are polyesters such as poly(glycolide-co-lactide) (PLGA), polyglycolic acid, poly-•-hydroxybutyrate, and polyacrylic acid ester. These particles have the additional advantage of protecting the agent from degradation by the body. These particles depending on their size, composition, and the agent being delivered can be administered to an individual using any route available (e.g., oral, injectional, inhalational administration).

Biocompatibility is of special importance when a sustained release vehicle such as microparticles is used for local delivery of an agent to sensitive or vital structures (e.g., nerves, eyes, atria, uterus), particularly if the dwell time of the polymeric device in the target tissue is much longer than the clinical efficacy of the delivered drug.

The transfer of genetic information to cells in the form of nucleic acids offers the potential for broad application in medicine. Many techniques have been studied for delivering nucleic acids to cells (Goodman & Gilman's The Pharmacological Basis of Therapeutics Ninth Edition, Hardman and Limbird, eds., McGraw-Hill, New York, 1996, pages 77-101; incorporated herein by reference). These include administering naked (unencapsulated) DNA, DNA encapsulated in poly(lactic-co-glycolic) acid (PLGA) and modified PLGA, and DNA encapsulated in chitosan microspheres (Hedley et al. “Microspheres containing plasmid-encoded antigens elicit cytotoxic T-cell responses” Nat. Med. 4(3):365-368, 1998; Roy et al. “Oral gene delivery with chitosan-DNA nanoparticles generates immunologic protection in a murine model of peanut allergy” Nat. Med. 5(4):387-391, 1999; Walter et al. “Microencapsulation of DNA using poly(DL-lactide-co-glycolide): stability issues and release characteristics” J. Controlled Release 61(3):361-374, 1999; each of which is incorporated herein by reference). One of the more promising methods of delivering nucleic acids takes advantage of viruses' natural ability to deliver nucleic acids (e.g., DNA, RNA) into cells.

What is needed is a drug delivery vehicle that will provide delivery of a polynucleotide without degradation of the polynucleotide, will provide high rates of transfection, will not lead to inflammatory reactions, and is biocompatible with the tissue to which the polynucleotide is being delivered.

### SUMMARY OF THE INVENTION

The present invention provides a system for delivering a polynucleotide encapsulated in a lipid-protein-sugar matrix to an individual. The encapsulated polynucleotide may be DNA, RNA, a hybrid of DNA and RNA, or a derivative thereof. In a preferred embodiment, the agent is encapsulated in the lipid-protein-sugar matrix to form small particles for administration to the individual. Typically the size of these particles ranges from 5 micrometers to 50 nanometers. In a particularly preferred embodiment, the particles are small enough to be taken up by cells (e.g., by phagocytosis, endocytosis, pinocytosis). The lipid-protein-sugar particles (LPSP) typically comprise a surfactant or phospholipid or similar hydrophic or amphiphilic molecule; a protein; a simple and/or complex sugar; and the polynucleotide to be delivered. In a particularly preferred embodiment, the lipid is dipalmitoylphosphatidylcholine (DPPC), the protein is albumin, and the sugar is lactose. In another particularly preferred embodiment, a synthetic polymer is substituted for at least one of the components of the LPSPs-lipid, protein, and/or sugar. One advantage of LPSPs over other polymeric vehicles is that the compounds used to create LPSPs are naturally occurring and therefore have improved biocompatibility compared to other polymers such as poly(lactic-co-glycolic acid) (PLGA). The LPSPs may be prepared using techniques known in the art including spray drying.

In another aspect, the present invention provides a method of administering the inventive LPSPs and pharmaceutical compositions comprising LPSPs to an individual human or animal. The LPSPs once prepared can be administered to the individual by any means known in the art including, for example, intravenous injection, intradermal injection, rectally, orally, intravaginally, inhalationally, etc. Preferably, administration of the encapsulated agent provides release of the polynucleotide into the cell.

In yet another aspect, the present invention provides a method of transfecting cells with a polynucleotide using LPSPs as the delivery vehicle. An effective amount of the inventive particles containing the polynucleotide to be delivered are contacted with cells so that the polynucleotide is delivered into the cells. In a particularly preferred embodiment, the cells are stems cells such as embryonic stem cells, hematopoietic stem cells, hepatic stem cell, neural stem cell, etc. In another particularly preferred embodiment, the cells are derived from a patient with a condition to be treated by isolating cells, transfecting them, and administering them back to the patient. In certain preferred embodiments, the transfection results in long-term gene expression.

### Definitions

“Adjuvant”: The term adjuvant refers to any compound which is a nonspecific modulator of the immune response. In certain preferred embodiments, the adjuvant stimulates the immune response. Any adjuvant may be used in accordance with the present invention. A large number of adjuvant compounds is known; a useful compendium of many such compounds is prepared by the National Institutes of Health and can be found on the world wide web (http:/www.niaid.nih.gov/daids/vaccine/pdf/compendium.pdf, incorporated herein by reference; see also Allison Dev. Biol. Stand. 92:3-11, 1998; Unkeless et al. Annu. Rev. Immunol. 6:251-281, 1998; and Phillips et al. Vaccine 10: 151-158,1992, each of which is incorporated herein by reference).

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 20% cell death and do not induce inflammation or other such adverse effects in vivo.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed). In certain preferred embodiments, the chemical reactions relied upon to break down the biodegradable compounds are uncatalyzed.

“Effective amount”: In general, the “effective amount” of an active agent or LPSPs refers to the amount necessary to elicit the desired biological response. As will be appreciated by those of ordinary skill in this art, the effective amount of LPSPs may vary depending on such factors as the desired biological endpoint, the agent to be delivered, the composition of the encapsulating matrix, the target tissue, etc. For example, the effective amount of LPSPs containing a local anesthetic to be delivered to provide a nerve block is the amount that results in a reduction in sensation of a desired area for a desired length of time. In another example, the effective amount of LPSPs containing an antigen to be delivered to immunize an individual is the amount that results in an immune response sufficient to prevent infection with an organism having the administered antigen.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

“Sugar”: The term “sugar” refers to any carbohydrate. Sugars useful in the present invention may be simple or complex sugars. Sugars may be monosaccharides (e.g., dextrose, fructose, inositol), disaccharides (e.g., sucrose, saccharose, maltose, lactose), or polysaccharides (e.g., cellulose, glycogen, starch). Sugars may be obtained from natural sources or may be prepared synthetically in the laboratory. In a preferred embodiment, sugars are aldehyde- or ketone-containing organic compounds with multiple hydroyxl groups.

“Surfactant”: Surfactant refers to any agent which preferentially absorbs to an interface between two immiscible phases, such as the interface between water and an organic solvent, a water/air interface, or an organic solvent/air interface. Surfactants usually possess a hydrophilic moiety and a hydrophobic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent. The terms surfactant and emulsifier may be used interchangeably with the term lipid in the present application.

### BRIEF DESCRIPTION OF THE DRAWING

FIG. 1 is a scanning electron micrograph of spray-dried lipid-protein particles (60% dipalmitoylphosphatidylcholine) as prepared.

FIG. 2 shows the cumulative release from a dialysis tube of bupivacaine encapsulated in 10% (w/w) bupivacaine lipid-protein particles with 60% (•) or 99% (•) of the excipients being dipalmitoylphosphatidylcholine, or an equivalent amount of 0.5% (w/v) bupivacaine in solution (•). Also shown is release from 50% (w/w) bupivacaine PLGA microsphere (◯). Data shown are means with standard deviations. n=4 for all points.

FIG. 3 shows the comparison of the durations of sensory and motor blockade for 10% (w/w) bupivacaine lipid-protein (•), 50% (w/w) bupivacaine PLGA microspheres (◯), and 0.5% (w/v) bupivacaine in solution (•). Points falling above the diagonal line bisecting the graph represent a relative sensory predominance in nerve blockade, while those falling below have motor predominance.

FIG. 4 shows the time course of thermal latency in the uninjected leg following sciatic nerve block in animals injected with 10% (w/w) bupivacaine lipid-protein particles (•) and in animals injected with 50% (w/w) bupivacaine PLGA microspheres (◯). Here thermal latency in the uninjected (contralateral) leg is used as a measure of systemic drug distribution. Data shown are means with standard deviations. None of the differences in latency between the two groups were statistically significant.

FIG. 5 shows a photomicrograph of a control nerve at low power (A). Note the absence of inflammatory cells outside the neural sheath (perineurium). FIG. 5B shows a high power view (400×) of connective tissue outside the perineurium. Note the looseness and low cellularity of the connective tissue.

FIG. 6 shows PLGA microspheres at the sciatic nerve. The microspheres are seen in close proximity to the nerve, but outside the nerve sheath. N=sciatic nerve. BV=blood vessel. M=muscle. MS=microspheres.

FIG. 7 shows a summary of histological findings over time. FIG. 7A shows dissection scores. Median+/−25th and 75th percentiles. No statistically significant difference between the groups was observed. FIG. 7B shows the prevalence of particle residue. FIG. 7C shows areas of inflammation at two weeks. Median+/−25th and 75th percentiles. In the PLGA group, the area was too large and amorphous at 4 days to be dissected en bloc. At 7 months there was no obvious entity to measure in either group. FIG. 7D shows the cell density of the inflammatory response. Data shown are means with standard deviations. In summary, there were robust differences at two weeks post injection.

FIG. 8 shows photomicrographs four days after particle injection. FIG. 8A shows the injection of PLGA microspheres. The microspheres are surrounded by a dense infiltrate, surrounded by intense granulomatous inflammation, with some foreign body giant cells. In general (and at all time points examined), the inflammatory response to PLGA microspheres was confined to clearly demarcated pockets that were firm and slightly gritty to palpation. FIG. 8B shows injection of LPS PS. A dense inflammatory reaction that was more diffuse than that resulting from the injection of PLGA microspheres was observed. Pockets of particle were smaller and softer to the touch. In both cases, inflammation also involved adjacent muscle and epineural adipose tissue. The injection site had necrotic muscle fibers, myophagocytosis, and myocyte regeneration—all signs of acute muscle injury.

FIG. 9 shows photomicrographs two weeks after particle injection. FIG. 9A shows the injection of PLGA microspheres. The histological appearance was very similar to that at 4 days (and at 8 weeks), with lymphocytes, macrophages, and giant cells in granulomas. The asterisk denotes a “ghost” of a microsphere. One sample in this group had active myositis. FIG. 9B shows the injection of LPSPs. There was a small loose, predominantly lymphocytic infiltrate. In both cases, histological appearance was the same for particles without drug.

FIG. 10 shows ectopic particles. FIG. 10A shows sub-perineurial microspheres. Arrows indicate “ghosts” of microspheres. FIG. 10B shows a pocket of microspheres approximately 0.4 mm×4 mm×8 mm found at the knee of a rat (about 2.5 cm from site of injection).

FIG. 11 shows a photograph of an agarose gel under UV illumination. The gel shows DNA that has been encapsulated in LPSPs and later extracted and purified from the particles. The DNA is largely undamaged during the encapsulation procedure used to form the LPSPs.

### DETAILED DESCRIPTION OF CERTAIN PREFERRED EMBODIMENTS OF THE INVENTION

The present invention provides a system including a pharmaceutical composition of lipid-protein-sugar particles (LPSP) containing a polynucleotide as well as methods of preparing and administering the LPSPs. Preferably, the polynucleotide is delivered into the cell. The matrix of these LPSPs also are preferably substantially biocompatible and preferably cause minimal inflammatory reaction, and the degradation products are preferably easily eliminated by the body (i.e., the components of the LPSPs matrix are biodegradable).

Polynucleotide

The polynucleotide to be delivered in the LPSPs may be any nucleic acid including but not limited to RNA and DNA. The polynucleotides may be of any size or sequence, and they may be single- or double-stranded. In certain preferred embodiments, the polynucleotide is greater than 100 base pairs long. In certain other preferred embodiments, the polynucleotide is greater than 1000 base pairs long and may be greater than 10,000 base pairs long. The polynucleotide is preferably purified and substantially pure. Preferably, the polynucleotide is greater than 50% pure, more preferably greater than 75% pure, and most preferably greater than 95% pure. The polynucleotide may be provided by any means known in the art. In certain preferred embodiments, the polynucleotide has been engineered using recombinant techniques (for a more detailed description of these techniques, please see Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989); each of which is incorporated herein by reference). The polynucleotide may also be obtained from natural sources and purified from contaminating components found normally in nature. The polynucleotide may also be chemically synthesized in a laboratory. In a preferred embodiment, the polynucleotide is synthesized using standard solid phase chemistry.

The polynucleotide may be modified by chemical or biological means. In certain preferred embodiments, these modifications lead to increased stability of the polynucleotide. Modifications include methylation, phosphorylation, end-capping, etc.

Derivatives of polynucleotides may also be used in the present invention. These derivatives include modifications in the bases, sugars, and/or phosphate linkages of the polynucleotide. Modified bases include, but are not limited to, those found in the following nucleoside analogs: 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine. Modified sugars include, but are not limited to, 2′-fluororibose, ribose, 2′-deoxyribose, 3 ′-azido-2′, 3′-dideoxyribose, 2′, 3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphorarnidite linkages. Combinations of the various modifications may be used in a single polynucleotide. These modified polynucleotides may be provided by any means known in the art; however, as will be appreciated by those of skill in this art, the modified polynucleotides are preferably prepared using synthetic chemistry in vitro.

The polynucleotides to be delivered may be in any form. For example, the polynucleotide may be a circular plasmid, a linearized plasmid, a cosmid, a viral genome, a modified viral genome, an artificial chromosome, etc.

The polynucleotide may be of any sequence. In certain preferred embodiments, the polynucleotide encodes a protein or peptide. The encoded proteins may be enzymes, structural proteins, receptors, soluble receptors, ion channels, pharmaceutically active proteins, cytokines, interleukins, antibodies, antibody fragments, antigens, coagulation factors, albumin, growth factors, hormones, insulin, etc. The polynucleotide may also comprise regulatory regions to control the expression of a gene. These regulatory regions may include, but are not limited to, promoters, enhancer elements, repressor elements, TATA box, ribosomal binding sites, stop site for transcription, etc. In other particularly preferred embodiments, the polynucleotide is not intended to encode a protein. For example, the polynucleotide may be used to fix an error in the genome of the cell being transfected.

The polynucleotide may also be provided as an antisense agent. Antisense therapy is meant to include, e.g., administration or in situ provision of single- or double-stranded oligonucleotides or their derivatives which specifically hybridize, e.g., bind, under cellular conditions, with cellular mRNA and/or genomic DNA, or mutants thereof, so as to inhibit expression of the encoded protein, e.g., by inhibiting transcription and/or translation (Crooke “Molecular mechanisms of action of antisense drugs” Biochim. Biophys. Acta 1489(1):31-44, 1999; Crooke “Evaluating the mechanism of action of antiproliferative antisense drugs” Antisense Nucleic Acid Drug Dev. 10(2):123-126, discussion 127, 2000; Methods in Enzymology volumes 313-314, 1999; each of which is incorporated herein by reference). The binding may be by conventional base pair complementarity, or, for example, in the case of binding to DNA duplexes, through specific interactions in the major groove of the double helix (i.e., triple helix formation) (Chan et al. J. Mol. Med. 75(4):267-282, 1997; incorporated herein by reference).

The polynucleotide may be associated with other agents in the LPSPs. Such agents include poly-amines which neutralize the negative charge in the phosphate backbone of the polynucleotide. These agents are thought to allow for the passage of the neutral complex through cellular and nuclear membranes. These agents may also protect the polynucleotide from degradation once the polynucleotide is in the cell.

In a particularly preferred embodiment, the polynucleotide to be delivered comprises a sequence encoding an antigenic peptide or protein. LPSPs containing these polynucleotides can be delivered to an individual to induce an immunologic response sufficient to decrease the chance of a subsequent infection and/or lessen the symptoms associated with such an infection. The polynucleotide of these vaccines may be combined with interleukins, interferon, cytokines, and adjuvants such as cholera toxin, alum, Freund's adjuvant, etc. A large number of adjuvant compounds is known; a useful compendium of many such compounds is prepared by the National Institutes of Health and can be found on the world wide web (http:/www.niaid.nih.gov/daids/vaccine/pdf/compendium.pdf, incorporated herein by reference; see also Allison Dev. Biol. Stand. 92:3-11, 1998; Unkeless et al. Annu. Rev. Immunol. 6:251-281, 1998; and Phillips et al. Vaccine 10:151-158,1992, each of which is incorporated herein by reference).

The antigenic protein or peptides encoded by the polynucleotide may be derived from such bacterial organisms as Streptococccus pnuemoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; from such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; and from such fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodiumfalciparum, Trypanosoma brucei, Entamoeba histolytica, Toxoplasma gondii, Trichomonas vaginalis, Schistosoma mansoni, and the like.

Microparticle Excipients

The agent is preferably encapsulated in a matrix comprising lipid, protein, and sugar to form microparticles. In a preferred embodiment, the diameter of the microparticles is less than 10 micrometers and more preferably less than 5 micrometers. The size of the microparticles and distribution of sizes may be selected by one of ordinary skill in the art based on the agent being delivered, the target tissue, route of administration, method of uptake by the cells, etc. In certain embodiments, one of the three components may be eliminated from the matrix. In other embodiments, a synthetic polymer (e.g., poly(lactic-co-glycolic acid) (PLGA), polyglycolic acid (PGA), polesters, polyanhydrides, polyamides, etc.) is used as a substitute for at least one of the components of the LPSPs. The specific ratios of the excipients may range widely depending on factors including size of particle, porosity of particle, agent to be delivered, desired agent release profile, target tissue, etc. One of ordinary skill in the art may test a variety of ratios and specific components to determine the composition correct for the desired purpose. Any known lipid, protein, and sugar, natural or unnatural, may be used to prepare the inventive microparticles.

Lipids or Surfactants or Emulsifiers

The lipid portion of the matrix of the inventive LPSPs is thought to bind the particle together. The hydrophobicity of the lipid may also contribute to the slow release of the encapsulated drug. The percent of lipid in the matrix (excluding the agent) may range from 0% to 99%, more preferably from 3% to 99%.

Any lipid, surfactant, or emulsifier known in the art is suitable for use in making the inventive microparticles. Such surfactants include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid amides; sorbitan trioleate (Span 85) glycocholate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; and phospholipids. The lipid component may also be a mixture of different lipid molecules. These lipid may be extracted and purified from a natural source or may be prepared synthetically in a laboratory. In a preferred embodiment, the lipids are commercially available.

Protein

The protein component of the encapsulating matrix may be any protein or peptide. The protein of the LPSPs presumably plays a structural role in the microparticles. Proteins useful in the inventive system include albumin, gelatin, whole cell extracts, antibodies, and enzymes. In certain preferred embodiments, the protein may be chosen based on a known interaction between the protein and the polynucleotide being delivered. For example, a polynucleotide containing a binding site for a protein transcription factor may be delivered along with that transcription factor. The percentage of protein in the matrix (excluding the agent to be delivered) may range from 0% to 99%, more preferably 1% to 80%, and most preferably from 1% to 60%.

In certain preferred embodiments, the polynucleotide is delivered together with a protein agent. In these embodiments, the protein agent to be delivered may make up all or a portion of the protein component of the encapsulating matrix. Preferably, the protein maintains a significant portion of its original activity after having been processed to form microparticles

In another particularly preferred embodiment, at least a portion of the protein is immunoglobulins or fragments thereof. These immunoglobulins may serve as a targeting agent. For example, the binding site of the immunoglobulin may be directed to an epitope normally found in a tissue or on the cell surface of cells being targeted. The targeting of a specific receptor may lead to endocytosis of the microparticle. For example, the antibody may be directed to the LDL receptor.

The protein component may be provided using any means known in the art. In certain preferred embodiments, the protein is commercially available. The protein may also be purified from natural or recombinant sources, or may be chemically synthesized. In certain preferred embodiments, the protein has been purified and is 75% pure, more preferably 90% pure.

Sugar

The sugar component of the LPSPs may be any simple or complex sugar. The sugar component of the matrix is thought to play a structural role in the particles and may also lead to increased biocompatibility. The percentage of sugar in the LPSP matrix excluding the agent can range from 0% to 99%, more preferably from approximately 0.5% to approximantely 50%, and most preferably from approximately 0.5% to approximately 40%.

Natural as well as unnatural sugars may be used in the inventive LPSPs. Sugars that may be used in the present invention include, but are not limited to, galactose, lactose, glucose, maltose, starches, cellulose and its derivatives (e.g., methyl cellulose, carboxymethyl cellulose, etc.), fructose, dextran and its derivatives, raffinose, mannitol, xylose, dextrins, glycosaminoglycans, sialic acid, hyaluronic acid, chitosan, and chondroitin sulfate. Preferably, the sugar component like the protein and lipid components is biocompatible and biodegradable. In certain preferred embodiments, the sugar component is a mixture of sugars. The sugar may be from natural sources or may be synthetically prepared. Preferably, the sugar is commercially available.

In a particularly preferred embodiment, the sugar of the matrix may also function as a targeting agent. For example, the ligand of a receptor found on the cell surface of cells being targeted or a portion of the ligand may be the same sugar in the LPSP or may be similar to the sugar in the LPSP, or in a particularly preferred embodiment, the sugar may also be designed to mimic the natural ligand of the receptor.

Targeting Agents

The inventive LPSPs may be modified to include targeting agents since it is often desirable to target a LPSP to a particular cell, collection of cells, or tissue. A variety of targeting agents that direct pharmaceutical compositions to particular cells are known in the art (see, for example, Cotten et al, Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, etc. If the targeting agent is included throughout the particle, the targeting agent may be included in the mixture that is spray dried to form the particles. If the targeting agent is only on the surface, the targeting agent may be associated with (i.e., by covalent, hydrophobic, hydrogen boding, van der Waals, or other interactions) the formed particles using standard chemical techniques.

Pharmaceutical Compositions

Once the LPSPs have been prepared, they may be combined with other pharmaceutical excipients to form a pharmaceutical composition. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., LPSPs), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the LPSPs are suspended in a carrier fluid comprising 1% (w/v) sodium carboxymethyl cellulose and 0. 1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the LPSPs with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the LPSPs are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include ointments, pastes, creams, lotions, gels, powders, solutions, sprays, inhalants, or patches. The LPSPs are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the LPSPs of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the LPSPs of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the LPSPs in a proper medium. Absorption enhancers can also be used to increase the flux of the compound across the skin. The rate can be controlled by either providing a rate controlling membrane or by dispersing the LPSPs in a polymer matrix or gel.

Methods of Making Microparticles

The inventive microparticles may be prepared using any method known in this art. These include spray drying, single and double emulsion solvent evaporation, solvent extraction, phase separation, simple and complex coacervation, and other methods well known to those of ordinary skill in the art. A particularly preferred method of preparing the particles is spray drying. The conditions used in preparing the microparticles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix.

Methods developed for making microparticles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

If the particles prepared by any of the above methods have a size range outside of the desired range, the particles can be sized, for example, using a sieve.

As mentioned above, LPSPs are preferably prepared by spray drying. Prior methods of spray drying, such as those disclosed in PCT WO 96/09814 by Sutton and Johnson (incorporated herein by reference), provide the preparation of smooth, spherical microparticles of a water-soluble material with at least 90% of the particles possessing a mean size between 1 and 10 micrometers. The method disclosed by Edwards et al. in U.S. Pat. No. 5,985,309 (incorporated herein by reference) provides rough (non-smooth), non-spherical microparticles that include a water-soluble material combined with a water-insoluble material. Any of the methods described above may be used in preparing the inventive LPSPs. Specific methods of preparing LPSPs containing bupivacaine are described below in the Examples.

The method of preparing the LPSPs may be modified when a polynucleotide is to be encapsulated. Due to the fragility of polynucleotides and the desire to have them delivered undamaged, such factors as temperature, agitation, pH, solvent, and choice of excipients may need to be modified to encapsulate the polynucleotide in LPSPs. These modifications would be appreciated by one of skill in this art.

Administration

The LPSPs and pharmaceutical compositions containing LPSPs may be administered to an individual via any route known in the art. These include, but are not limited to, oral, sublingual, nasal, intradermal, subcutaneous, intramuscular, rectal, vaginal, intravenous, intraarterial, and inhalational administration. As would be appreciated by one of skill in this art, the route of administration is determined by the polynucleotide being administered, the target organ, the preparation being administered, time course of administration, disease being treated, etc.

Cell Transfection

The inventive LPSPs containing polynucleotides may also be used in the transfection of cells. In certain embodiments, the transfection is performed in vitro. The cells are contacted with an amount of LPSPs sufficient to allow transfection of the encapuslated DNA. Other agents may be added to the suspensions of LPSPs to effect the uptake of the particles by the cells. The cells to be transfected may be any cell such as those derived from an established cell line, from a biopsy, from a surgical specimen, from a bone marrow donation, etc. The cells may be bacterial, fungal, plant, or animal. In a preferred embodiment, the cells are derived from animals, and in certain embodiments the cells are human cells.

These and other aspects of the present invention will be further appreciated upon consideration of the following Examples, which are intended to illustrate certain particular embodiments of the invention but are not intended to limit its scope, as defined by the claims.

### EXAMPLES

### Example 1

Sciatic Nerve Blockade with Lipid-Protein-Sugar Particles Containing Bupivacaine

In this Example, the production and characterization of LPSPs in vitro is discussed along with the assessment of the in vivo local anesthetic efficacy of an optimal formulation in sciatic nerve blockade in the rat, using a neurobehavioural paradigm (Kohane et al. “A re-examination of tetrodotoxin for prolonged anesthesia” Anesthesiology 89:119-131, 1998; Thalhammer et al. “Neurologic evaluation of the rat during sciatic nerve block with lidocaine” Anesthesiology 82:1013-1025, 1995; each of which is incorporated herein by reference) that examines sensory (thermal nociception) and motor (weight bearing) function. The marked methodological variability between published reports on the in vivo effectiveness of controlled release local anesthetic preparations makes comparisons between them difficult. For this reason the LPSP delivery system is compared to another delivery system in a series of experiments where the particles are delivered by the same means, to the same location, in equal quantity, and where the neurobehavioural outcomes are evaluated by the same methods and at the same time intervals in a blinded manner. Large (20 to 120 μm) poly(lactic-co-glycolic) acid (PLGA)-based particles have been selected for this comparison because a) they have a long track-record of experimental use for anesthesia in the peripheral nervous system and spinal cord (Castillo et al. “Glucocorticoids prolong rat sciatic nerve blockade in vivo from bupivacaine microspheres” Anesthesiology 85:1157-1166, 1996; Curley et al. “Prolonged regional nerve blockade. Injectable biodegradable bupivacaine/polyester microspheres” Anesthesiology 84:1401-1410, 1996; Drager et al. “Prolonged intercostal nerve blockade in sheep using controlled release bupivacaine and dexamethasone from polyester microspheres” Anesthesiology 89:969-979, 1998; Estebe et al. “Prolongation of spinal anesthesia with bupivacaine-loaded (DL-lactide) microspheres” Anesth. Analg. 81:99-103, 1995; Le Corre et al. “Preparation and characterization of bupivacaine-loaded polylactide and polylactide-coglycolide microspheres” Int. J. Pharnaceut. 107:41-49, 1994; Le Corre et al. “In vitro controlled release kinetics of local anaesthetics from poly(D,L-lactide) and poly(lactide-co-glycolide) microspheres” J. Microencaps. 14:243-255, 1997; Wakiyama et al. “Preparation and evaluation in vitro of polylactic acid microspheres containing local anesthetics” Chem. Pharm. Bull. 29:3363-3368, 1981; Wakiyama et al. “Preparation and evaluation in vitro and in vivo of polylactic acid microspheres containing dibucaine” Chem. Pharm. Bull. 30:3719-3727, 1982; each of which is incorporated herein by reference), and b) such microspheres have been described as producing very slow release of local anesthetics (Curley et al. “Prolonged regional nerve blockade. Injectable biodegradable bupivacaine/polyester microspheres” Anesthesiology 84:1401-1410, 1996; incorporated herein by reference).

### Materials and Methods

Materials

Bupivacaine hydrochloride, human serum albumin (Fraction V), and lactose β-monohydrate were purchased from Sigma Chemical Co. (St. Louis, Mo.), L-α-dipalmitoylphosphatidylcholine (DPPC) from Avanti Polar Lipids (Alabaster, Ala.), poly (lactic-co-glycolic) acid (65:35, MW 110,000) (PLGA) from Medisorb, poly (vinyl alcohol) (88% hydrolyzed, MW 20,000) from Polysciences (Warrington, Pa.), ethyl acetate and methylene chloride (both HPLC grade) from EM Sciences (Gibbstown, N.J.), and USP grade ethanol from Pharmco Products, Brookfield, Conn. Bupivacaine hydrochloride was made into the free base by alkaline precipitation and filtration. The ultraviolet absorbance spectrum from 200 nm to 300 nm, and a standard curve of absorbance at 272 nm vs. concentration were determined for each batch of the free base for quality control purposes.

Preparation of Spray-Dried Lipid-Protein Particles (LPSPs)

A 70:30 (v/v) ethanol:water solvent system was employed for solubilization and spray drying of excipients and bupivacaine. The solutions were prepared in the following manner: (i) the DPPC and bupivacaine free base were dissolved in a given amount of ethanol, (ii) the lactose and albumin were dissolved in a given amount of water (pH adjusted to 7.0), and (iii) the solutions were mixed immediately prior to spray drying. Solute concentrations ranged from 1 to 4 grams per liter. The proportion of albumin to lactose was kept constant in experiments where the DPPC content was changed.

Solutions were spray-dried using a Model 190 bench top spray drier (Büchi Co, Switzerland). The spray-drying parameters (inlet temperature, fluid flow rate, drying airflow rate, and aspirator pressure) were optimized based on the yield and size characteristics of both the blank (no bupivacaine) and the bupivacaine-containing particles. The optimized conditions were: inlet temperature=115 to 120° C., solution feed rate=12 to 14 ml/min, drying airflow rate =600 l/min, and aspirator pressure=−18 barr. These conditions typically resulted in outlet temperatures in the range of 50 to 55° C. for a given experimental run.

Particle Size and Bulk Density

A small amount of particles was dispersed in 20 ml of Isotoner (Coulter Corp., Miami, Fla.) and analyzed for size via a Coulter Multisizer (Coulter Electronics Ltd., Luton, U.K.) equipped with a 50 μm (for LPSPs) or 140 μm (for PLGA microspheres) orifice tube. Bulk mass density of the 60% DPPC powders was estimated using a Dual Platform Microprocessor Controlled Tap Density Tester (Vankel Technology Group, Cary, N.C.).

Scanning Electron Microscopy

The surface morphology of spray-dried particles was examined using a JEOL Model 6320 FV field emission scanning electron microscope (provided by the Massachusetts Institute of Technology Department of Materials Science and Engineering Electron Microscopy Center). Samples were mounted on stubs and coated with a layer of gold/palladium. Samples were scanned at voltages between 5 and 10 kV at a probe current setting of 3 and a working distance of 7 millimeters.

Bupivacaine Content of LPSPs

In order to determine the bupivacaine content of LPSPs, 10 mg of particles were agitated (Touch Mixer model 2332, Fisher Scientific, Pittsburgh, Pa.) for 20 seconds in 1 mil ethyl acetate. One-half ml of 0.1 N NaOH was then added, and the mixture was agitated for an additional 60 seconds. The suspension was centrifuged for 10 minutes at 14,000 rpm. One half ml of the upper organic layer was withdrawn, diluted with an equal volume of fresh ethyl acetate, and the absorbance at 272 nm was then measured (Cary 50 Bio UV-Visible Spectrophotometer, Varian, Australia) in a quartz cuvette (Hellma, Mullheim, Germany). Bupivacaine content was determined by comparison to a standard curve. Blank (no bupivacaine) LPSPs served as controls, and when processed in this manner had negligible absorbance at 272 nm. As an additional control we determined the amount of albumin that may have accompanied the bupivacaine in the ethyl acetate extraction (this was important because the two compounds have overlapping absorbance spectra), using a commercial kit (BCA Protein Assay Reagent Kit, Pierce Chemical Co., Rockford, Ill.). The amount of albumin was below the detection limit (<25 μg/ml), and therefore could not account for measured absorbances at 272 nm.

In Vitro Release of Bupivacaine from Microparticles

Fifty mg of LPSPs or PLGA microspheres were suspended in 1 ml phosphate buffered saline pH 7.4 at 37° C. and inserted into the lumen of a Spectra/Por 1.1 Biotech Dispodialyzer with an 8,000 MW cut-off. The dialysis bag was placed into a test tube with 12 ml PBS and incubated at 37° C. on a tilt-table (Ames Aliquot Mixer, Miles). At predetermined intervals, the dialysis bag was transferred to a test tube with fresh PBS. The bupivacaine concentration in the dialysate was quantitated by measuring absorbance at 272 nm and referring to a standard curve. Observation of the entire spectrum, and performance of a protein assay (as above) confirmed the absence of albumin from the samples that were measured.

Preparation and Characterization of PLGA-Bupivacaine Microspheres

Microspheres loaded with 10% (w/w) and 50% (w/w) bupivacaine were prepared using a single emulsion method (Curley et al. “Prolonged regional nerve blockade. Injectable biodegradable bupivacaine/polyester microspheres” Anesthesiology 84:1401-1410, 1996; Watts et al. “Microencapsulation using emulsification/solvent evaporation: an overview of techniques and applications” Crit. Rev. Ther. Drug Carr. Sys. 7:235-259, 1990; each of which is incorporated herein by reference). Bupivacaine and PLGA were dissolved in methylene chloride, and the mixture was homogenized (Silverson L4R, Silverson Machines Ltd., Cheshire, England) in 50 ml 0.5% polyvinyl alcohol in 100 mM Trizma buffer pH 8.5 for 60 seconds. The resulting suspension was decanted into 100 ml of 0.05% polyvinyl alcohol in 100 mM Trisma pH 8.5 and stirred for 3 minutes prior to rotary evaporation (Büchi Rotavap, Büchi, Switzerland) in a 37° C. water bath until bubbling ceased. Spheres 20 μm to 120 μm in diameter were isolated by sieving (Newark Wire Co., Newark, N.J.), then resuspended in 50 ml of water. The suspension was washed three times by centrifugation at 5000 rpm for 5 minutes. The final pellet was lyophilized to dryness.

Bupivacaine content was determined by dissolving 10 mg of microspheres in 1 ml methylene chloride, and comparing the resulting UV absorbance at 272 nm to a standard curve. Under similar conditions, PLGA microspheres containing no bupivacaine showed negligible absorbance at 272 nm.

Animal Care

Young adult male Sprague-Dawley rats weighing 310-420 g each were obtained from Taconic Farms (Germantown, N.Y.), and housed in groups in a 6 AM-6 PM light-dark cycle. Animals were cared for in compliance with protocols approved by the Animal Care and Use Committee at the Massachusetts Institute of Technology, and the Principles of Laboratory Animal Care published by the National Institutes of Health. Rats were only injected once.

Sciatic Blockade Technique

Prior to nerve block injections, rats were anesthetized briefly (<2 minutes) with halothane. Concurrently, 75 mg of LPSPs or microspheres were suspended in 0.6 ml of carrier fluid (1% (w/v) sodium carboxymethyl cellulose, 0.1% (v/v) Tween 80) with gentle agitation (<5 sec) in preparation for injection. A 20G needle was introduced postero-medial to the greater trochanter, pointing in an anteromedial direction (Thalhammer et al. “Neurologic evaluation of the rat during sciatic nerve block with lidocaine” Anesthesiology 82:1013-1025, 1995; incorporated herein by reference). Once bone was contacted, the needle was withdrawn 1 mm and the particle-containing solution was injected. The left leg was always used for blocks; the right served as control.

Assessment of Nerve Blockade

The effectiveness of block was measured at various time points, applying the methods of Thalhammer et al. (Thalhammer et al. “Neurologic evaluation of the rat during sciatic nerve block with lidocaine” Anesthesiology 82:1013-1025, 1995; incorporated herein by reference), or modifications thereof (Kohane et al. “A re-examination of tetrodotoxin for prolonged anesthesia” Anesthesiology 89:119-131, 1998; incorporated herein by reference).

Nociceptive block was assessed by a modified hotplate test (Masters et al. “Prolonged regional nerve blockade by controlled release of local anesthetic from a biodegradable polymer matrix” Anesthesiology 79:1-7, 1993; incorporated herein by reference). Hind paws were exposed in sequence (left then right) to a hot plate at 56° C. (Model 39D Hot Plate Analgesia Meter, IITC Inc., Woodland Hills, Calif.), and the time (latency) until paw withdrawal was measured by a stopwatch. Thermal latency is a measure of the intensity of analgesia. If the paw remained in contact for 12 seconds, it was removed by the experimenter to avoid injury to the animal or the development of hyperalgesia. This test was repeated three times for each rat at every time-point.

Motor strength was assessed by holding the rat with its posterior above a digital balance and allowing it to bear weight on one hind paw at a time. The maximum weight that the rat could bear without its ankle touching the balance was quantified.

Neurobehavioural Data Processing

The data for nociceptive block are reported in terms of thermal latency (intensity) and duration of block. The duration of thermal nociceptive block is the time required for thermal latency to return to a value of 7 seconds (which is 50% of maximal block when a baseline thermal latency of approximately 2 seconds is taken into account). The duration of motor block was defined as the time for weight bearing to return halfway to normal from maximal block.

Statistical Analysis

Data are reported as means with standard deviations. Comparisons between groups were made using Student's t-test. These tests were unpaired except when comparing sensory vs. motor blockade in the same rat.

### Results

Production and Characterization of Lipid-Protein-Sugar Particles (LPSPs)

The spray-drying process conditions were initially optimized (with respect to yield) for the production of blank excipient particles (60:20:20 DPPC:albumin:lactose), as discussed in the methods section. (The reported percentage of DPPC refers to the composition of the excipients, excluding the delivered drug.) These conditions also appeared to be satisfactory for the production of the 10% (w/w) bupivacaine particles with varying DPPC contents. The results obtained from typical runs are shown in Table 1.

TABLE 1
Characteristics of lipid-protein-sugar particles (LPSPs) and PLGA-based
microspheres

| | DPPC | Bupivacaine | | Yield^{b, c} | Median diameter^{b, d} | Bupivacaine^{b, e} |
|---|---|---|---|---|---|---|
| Microparticle | (%^{a}) | loading (%) | n | (%) | (μm) | (%) |
| LPSP | 3 | 10 | 5 | 40 ± 6 | 2.58 ± 0.22 | 8 ± 0.4 |
| | 60 | 0 | 5 | 25 ± 5 | 4.66 ± 0.33 | — |
| | 60 | 10 | 5 | 37 ± 3 | 4.44 ± 0.39 | 8.8 ± 0.7 |
| | 99 | 10 | 5 | 37 ± 7 | 1.73 ± 0.05 | 7.6 ± 0.8 |
| PLGA | 0 | 50 | 6 | 53 ± 15 | 59 ± 12 | 44 ± 5 |
| microsphere | | | | | | |

^{a}Percentage of the total mass of excipient.
^{b}Values indicated are means ± standard deviations.
^{c}The fraction of solutes recovered, as weight % of the total amount of solutes taken in preparation.
^{d}Volume-weighted.
^{e}Measured as described in Methods (above).

The volume of fifty milligrams of each formulation loaded into test tubes were measured, and their densities were calculated. The 60% DPPC particles were less dense (0.07±0.004 g/ml) than the 3% DPPC (0.24±0.025, p=0.0007) and 99% DPPC (0.14±0.017, p=0.003) particles. This difference in density was probably due to a difference in particle size (Table 1): the mean diameter of the 60% DPPC particles was greater than those of the 3% and 99% DPPC particles (p=0.00001 and 0.000006 respectively). The 3% and 99% DPPC particles consistently formed macroscopic balls when stored as a dry powder, while this was not seen with the 60% DPPC particles. The impression of aggregation was confirmed by the observation that the average diameter of 3% and 99% DPPC particles rose to 19.28±0.01 and 11.08±0.34 μm respectively over a period of 4 weeks storage in a dessicator, while those of 60% DPPC particles did not change. The bupivacaine content of the various LPSPs formulations was similar (p=n.s.).

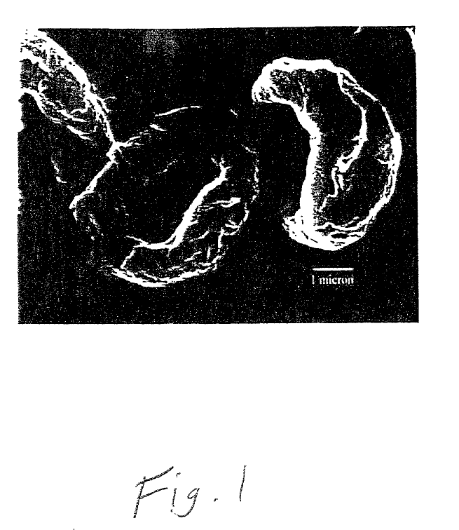

The 60% DPPC particles were spheroidal or, as can be seen in FIG. 1, concave in shape. Typical observed diameters were in the range of 3 to 5 microns. To ensure that the structural integrity of the particles was not impaired by the delivery method, we suspended 10 mg of particles in the carrier fluid (1% (w/v) carboxymethyl cellulose, 0.1% (v/v) Tween 80), mechanically agitated them for 10 seconds then injected them through a 20 G needle onto the inner wall of a test tube. Scanning electron microscopy demonstrated that the structure of the LPSPs was not altered (not shown).

Production and Characterization of PLGA-Bupivacaine Microspheres

PLGA microspheres produced as described in the methods section appeared to be regular spheres as viewed by light microscopy (images not shown), with diameters more than ten times larger than those of the LPSPs. Data from microsphere production are in Table 1. The final particle yield was comparable to that of the spray-dried particles. The data relevant to the production of the 10% (w/w) bupivacaine microspheres were similar to those for the 50% (w/w) microspheres, and their mean bupivacaine content (w/w) was 8% (n=2).

Bupivacaine Release from LPSPs

These experiments were performed so as to allow rational selection of a particular formulation for use in in vivo studies.

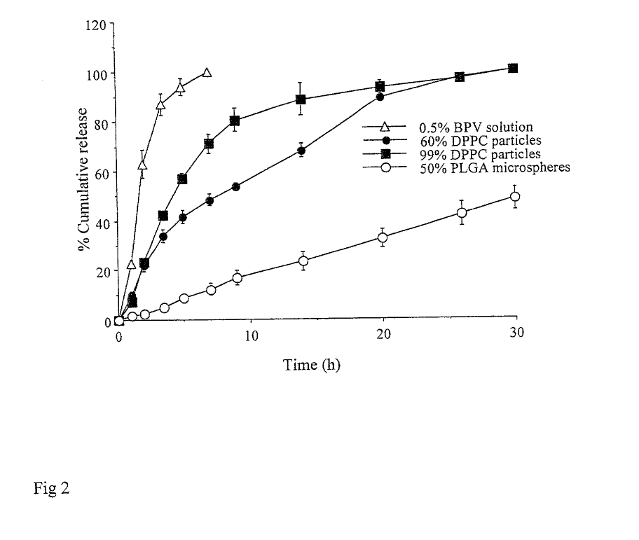

Pilot studies had shown that 3% DPPC particles almost completely disappeared by 18 hours after suspension in phosphate buffered saline, while 60% and 99% particles lasted many days. Consequently, we focused on the latter preparations. Bupivacaine release from 50 mg samples of 10% loaded (w/w) bupivacaine-LPSPs (n=4 for each particle formulation) was measured. FIG. 2 shows the cumulative release of bupivacaine over time. Both particle types caused delayed release of bupivacaine into the dialysate compared to the unencapsulated drug (1 ml of 0.5% (w/v) bupivacaine, or 5 mg). Both 60% and 99% DPPC particles completely released their bupivacaine content within 24 hours. However, release from the 60% DPPC particles was more gradual: at 9 hours, the 60% DPPC particles had released 53.8±1.5% of their bupivacaine content, whereas the 99% DPPC particles had released 80.6±4.7% (p=0.0002). Consequently, the 60% DPPC formulation was selected for in vivo studies. FIG. 2 also shows the release of bupivacaine from 50% (w/w) PLGA particles (n=4). The release, on a percentage basis, was much slower than that from LPSPs: less than 50% of total drug content was released in 30 hours, at which time the LPSPs had released 100% of drug content. The total amount of drug released by the LPSPs was slightly greater than that released by PLGA microspheres at most early time points (by 3.5 hours, the LPSPs had released 1.65±0.17 mg of bupivacaine vs. 1.26±0.15 mg for PLGA microspheres, p=0.01). This relationship was reversed at longer durations (by 9 hours the LPSPs had released 2.6±0.2 mg of bupivacaine, compared to 4.2±0.7 mg for the PLGA microspheres (p=0.02)).

Effectiveness of Sciatic Nerve Block

Rats were injected at the sciatic nerve with 75 mg (•215 mg/kg) of spray-dried LPSPs containing 10% (w/w) bupivacaine, and the time course of nerve blockade was followed. All rats injected with 10% (w/w) bupivacaine LPSPs achieved maximal nerve block (thermal latency =12 seconds) by the time of the first testing (30 minutes). Four out of ten rats injected with 50% bupivacaine microspheres did not achieve maximal block by that time. Nine out of ten rats injected with 50% (w/w) bupivacaine microspheres had maximal block by one hour after injection. All achieved maximal block within 3 hours.

The average duration of thermal nociceptive block from 10% (w/w) bupivacaine LPSPs was 468±210 min (n=10). The duration of thermal nociceptive block obtained from injection with 75 mg of PLGA microspheres with 50% (w/w) loading of bupivacaine was 706±344 min (n=10). This was not statistically different from the duration obtained with the 10% (w/w) bupivacaine LPSPs (p=0.08).

In order to compare the efficacy of equal loading with bupivacaine, rats were injected with 75 mg of 10% (w/w) bupivacaine PLGA microspheres (n=5), and 50% (w/w) bupivacaine LPSPs (n=2). The former did not result in nerve block as defined by our paradigm, while the latter caused rapid demise of the rats.

Six (6) rats were injected with 75 mg of blank DPPC-albumin-lactose particles in order to verify that the increased efficiency (comparable duration of block with much lower drug loading) of the LPSPs over bupivacaine microspheres was not due to an intrinsic nerve blocking-effect of the component excipients. Blank LPSPs did not produce any detectable nerve block.

Modality-Specificity of Nerve Blockade.

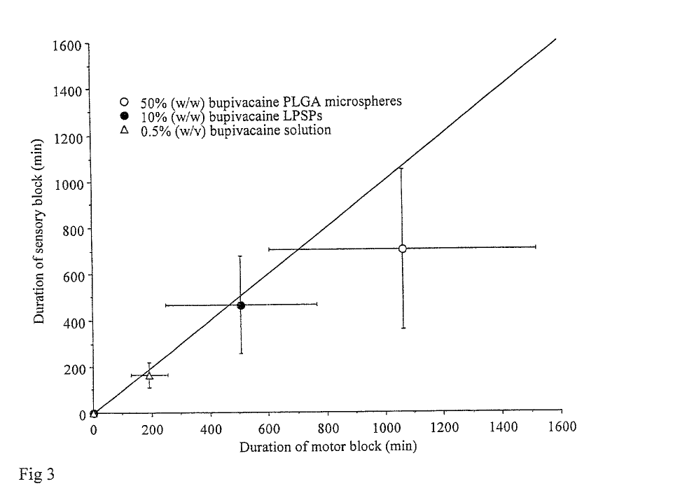

Blank LPSPs and 10% (w/w) bupivacaine microspheres did not cause any impairment in sensory or motor function. Motor blockade from 10% (w/w) bupivacaine LPSPs lasted 508±258 min, while that from 50% (w/w) bupivacaine microspheres lasted 1062±456 min (p=0.005). FIG. 3 focuses on the clinically important comparison of the durations of motor block (x-axis) and sensory block (y-axis) for both preparations. The motor block from the PLGA microsphere preparation lasted 50% longer than did the sensory block (p=0.003), as evidenced by the location of the representative point below the line of unity. The LPSPs had durations of sensory and motor block that were not statistically different from each other (8% difference, p=0.37).

Systemic Distribution of Bupivacaine.

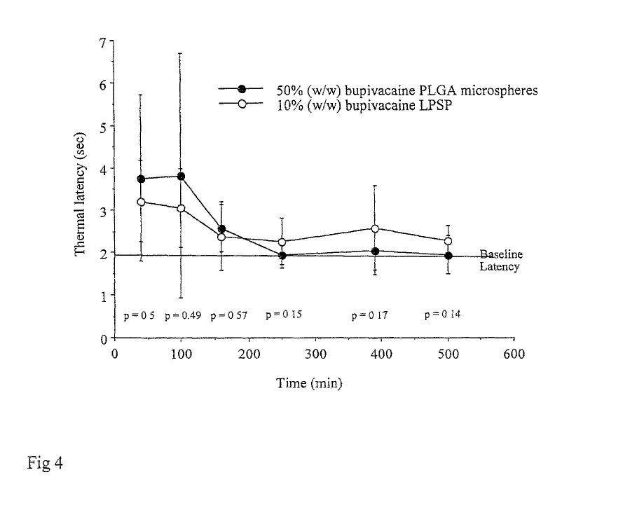

The presence of functional deficits in the un-injected extremity was used as a measure of the degree of systemically distributed local anesthetic (i.e., toxicity). Thermal latency (the length of time that a rat would leave his paw on the hotplate) was measured in the un-injected leg at predetermined intervals, in rats who received 10% bupivacaine LPSPs or 50% bupivacaine microspheres (FIG. 4). There was no statistically significant difference between the mean latencies in the two groups at any time point.

One rat (out of 11) injected with 50% bupivacaine microspheres died, approximately 2 hours after injection. Necropsy revealed congestion of the liver and kidneys, most consistent with heart failure. Both rats injected with 50% (w/w) bupivacaine LPSPs died. There were no deaths in the 10% (w/w) bupivacaine LPSP group (n=10), or 10% (w/w) PLGA microsphere group.

Encapsulation improved the safety and efficacy of bupivacaine. None of the rats injected with 10% (w/w) bupivacaine LPSPs had marked increases in contralateral latency. In comparison, rats (n=6) injected with an equivalent amount of bupivacaine in solution (1.5 ml of 0.5% bupivacaine, i.e. 7.5 mg) had a duration of block of 166±55 min. For this experiment larger rats (approx. 410 g) were used than those used in the remainder of the study, in order to avoid animal death (the median lethal dose of bupivacaine in adult rats is 30±5 mg/kg (Kohane et al. “Sciatic nerve blockade in infant, adolescent, and adult rats: a comparison of ropivacaine and bupivacaine” Anesthesiology 89:1199-1208, 1998; incorporated herein by reference), or 10.5 mg in a 350 g rat). Even so, one of those rats had severe signs of systemic toxicity (thermal latency=12 seconds in the uninjected leg). It was not possible to directly compare the efficacy of bupivacaine solution and 50% bupivacaine microspheres, since the dose of bupivacaine contained in 75 mg of those microspheres (38.5 mg) is approximately three times the median lethal dose of the unencapsulated drug (Kohane et al “Sciatic nerve blockade in infant, adolescent, and adult rats: a comparison of ropivacaine and bupivacaine” Anesthesiology 89:1199-1208, 1998). Nevertheless, it is obvious that the microspheres increased the safety of bupivacaine.

### Discussion

Of the three LPSP formulations tested in vitro, the 60% DPPC particles appeared optimal in terms of drug release of bupivacaine. The slower release of bupivacaine from the 60% DPPC particles compared to the 99% DPPC particles was somewhat surprising; a priori one might have expected the more hydrophobic nature of the latter particle type to delay release to a greater extent. This discrepancy may be related to the larger size of the 60% DPPC particles, which may impede access of water to the encapsulated drug and of drug to the exterior, or to a degree of bupivacaine binding by albumin.

The DPPC-albumin-lactose particles appear to be effective as vehicles for the local delivery of percutaneously injected local anesthetics in rats. The LPSPs had a more rapid onset of nerve block than the PLGA microspheres, which may be a reflection of the initial more rapid release of drug from the LPSPs. They were as effective as PLGA microspheres in terms of duration of local anesthesia, with one-fifth the initial loading of drug. (The duration of block that we obtained with the 50% bupivacaine microspheres is considerably longer than previously published values. Seventy-five percent loaded particles have been reported to last 6.0±3.0 hours (Curley et al. “Prolonged regional nerve blockade. Injectable biodegradable bupivacaine/polyester microspheres” Anesthesiology 84:1401-1410, 1996; incorporated herein by reference), compared to 11.8±5.7 hours for the 50% loaded particles in this study.) It would appear, based on the in vitro release studies, that this improved ratio of duration of block to drug loading most likely stems from the proportionally more rapid release of drug from the LPSPs. An alternative explanation would be that the LPSPs themselves have an effect on nerve function. While this possibility cannot be excluded, LPSPs without bupivacaine did not cause any detectable deficits in nerve function.

The more rapid fractional release of drug from the LPSPs did not result in greater toxicity than occurred from PLGA microspheres, as evidenced by the fact that thermal latency in the uninjected leg was not increased. (Increases in contralateral latency are an early sign of severe local anesthetic toxicity (Kohane et al. “Sciatic nerve blockade in infant, adolescent, and adult rats: a comparison of ropivacaine and bupivacaine” Anesthesiology 89:1199-1208, 1998; incorporated herein by reference).) The in vitro data suggest that this was because the discrepancy in total drug release between the two particle types was not as great as the fractional (percentage) difference. PLGA microspheres would appear to provide a better margin of safety at high bupivacaine loadings.

In general, it is not desirable for the motor block to be of longer duration than the sensory block (resulting in a paralyzed limb with full sensation). In fact, there are applications (such as obstetric anesthesia) where sensory block in the absence of motor block is desirable (so the mother can push during labor while still obtaining pain relief). The LPSPs had a more favorable ratio of duration of sensory to motor block than did the PLGA microspheres. The explanation for this difference is most likely to be pharmacokinetic. The large myelinated fibers (“A fibers”) that mediate motor function are more sensitive to amino-amide local anesthetics than are the small unmyelinated fibers that mediate pain (“C fibers”) (Wildsmith et al. “Differential nerve blocking activity of amino-ester local anaesthetics” Br. J. Anaesth. 57:612-620, 1985; incorporated herein by reference). Thus, one would expect sensation to return before motor function. In the case of the PLGA microspheres, the rate of decline of the local concentration of bupivacaine is probably slower, so that the time interval between the termination of sensory blockade and motor blockade is longer. The kinetic argument for the difference between the functional selectivities of LPSPs and PLGA microspheres is supported by the observation that bupivacaine solution (in the absence of any controlled release device) also shows approximately equal durations of sensory and motor block (FIG. 3), as has been previously noted in this animal model (Kohane et al. “Sciatic nerve blockade in infant, adolescent, and adult rats: a comparison of ropivacaine and bupivacaine” Anesthesiology 89:1199-1208, 1998; Kohane et al. “A re-examination of tetrodotoxin for prolonged anesthesia” Anesthesiology 89:119-131, 1998; each of which is incorporated herein by reference).

It bears mentioning that the LPSPs are of a size and density that makes them suitable for inhalational delivery. (The tap density—a more standardized measure of particle density—of the 60% DPPC LPSPs was 0.11±0.04 g/ml.) Nebulized local anesthetics have been used in a variety of roles in the management of medical problems of the upper airway and pulmonary tree (Keane et al. “Comparison of nebulized and sprayed topical anaesthesia for fiberoptic bronchoscopy” Eur. Respir. J. 5:1123-1125, 1992; incorporated herein by reference), including the management of asthma (Decco et al. “Nebulized lidocaine in the treatment of severe asthma in children: a pilot study” Ann. Allergy Asthma Immunol. 82:29-32, 1999; Hunt et al. “Effect of nebulized lidocaine on severe glucocorticoid-dependent asthma” Mayo Clin. Proc. 71:361-368, 1996; incorporated herein by reference). Nebulized lidocaine results in lower serum levels of drug than are achieved by equieffective intravenous doses (Groeben et al. “Both intravenous and inhaled lidocaine attenuate reflex bronchoconstriction but at different plasma concentrations” Am. J. Respir. Crit. Care Med. 159:530-535, 1999; incorporated herein by reference). Thus it is conceivable that particles of this sort could be beneficial in severe asthma, or in blunting patient responses to intratracheal suctioning, bronchoscopy, and other noxious procedures.

In summary, controlled release of bupivacaine using lipid-protein-sugar particles can provide prolonged duration local anesthesia that is as effective (depth and duration of anesthesia) as that conferred by conventional polymer-based particles.

### Example 2

Biocompatibility of Lipid-Protein-Sugar Particles Containing Bupivacaine in the Perineurium

In this second Example, the potential for a number of possible adverse reactions to the injected lipid-protein-sugar particles is assessed. The primary focus of the study was an examination of the biocompatibility of particles in terms of inflammatory response and gross neural injury as assessed by standard histological techniques. The incidence of “touch-evoked agitation” was also examined. This phenomenon was described in animals that received intrathecal injections of liposomes and phospholipid emulsions containing local anesthetics (Yanez et al. “Touch-evoked agitation produced by spinally administered phospholipid emulsion and liposomes in rats” Anesthesiology 82:1189-1198, 1995; incorporated herein by reference): they appeared in distress when the injected area was palpated. Although this phenomenon is not well understood, it is may be due to a product of phospholipid hydrolysis and to be particularly prominent in phospholipids whose gel-transition temperatures are close to body temperature (Yanez et al. “Touch-evoked agitation produced by spinally administered phospholipid emulsion and liposomes in rats” Anesthesiology 82:1189-1198, 1995; incorporated herein by reference). Because of the unknown potential for nerve injury from these particles, the rats were also tested for the development of self-mutilation (“autotomy”) in the blocked leg (Wall et al. “Autotomy following peripheral nerve lesions: experimental anaesthesia dolorosa” Pain 7:103-111, 1979; incorporated herein by reference), a behavior that results from nerve injury and is believed to be pain-related (although this is controversial). PLGA microspheres per se do not produce autotomy (Xiao et al. “Prolonged sciatic nerve blockade prevents neither the rise in GAP-43 expression nor the development of pain-related behaviour following nerve crush injury” Anesthesiology 87:734A, 1997; incorporated herein by reference) when injected at the perineurium.

Evaluation of the relative biocompatibility of different controlled release preparations described in the literature is impeded by the marked heterogeneity of experimental designs. Therefore, we compare LPSP to another delivery vehicle that is commonly employed to deliver local anesthetics, microspheres made from high molecular weight poly(lactic-co-glycolic) acid (PLGA) (Castillo et al. “Glucocorticoids prolong rat sciatic nerve blockade in vivo from bupivacaine microspheres” Anesthesiology 85:1157-66, 1996; Curley et al. “Prolonged regional nerve blockade. Injectable biodegradable bupivacaine/polyester microspheres” Anesthesiology 84:1401-1410, 1996; Drager et al. “Prolonged intercostal nerve blockade in sheep using controlled release bupivacaine and dexamethasone from polyester microspheres” Anesthesiology 89: 969-979, 1998; Le Corre et al. “Preparation and characterization of bupivacaine-loaded polylactide and polylactide-coglycolide microspheres” Int. J. Pharmaceut. 107:41-49, 1994; Le Corre et al. “In vitro controlled release kinetics of local anaesthetics from poly(D,L-lactide) and poly(lactide-co-glycolide) microspheres” J. Microencaps. 14:243-255, 1997; Estebe et al. “Prolongation of spinal anesthesia with bupivacaine-loaded (DL-lactide) microspheres” Anesth. Analg. 81:99-103, 1995; Wakiyama et al. “Preparation and evaluation in vitro of polylactic acid microspheres containing local anesthetics” Chem. Phann. Bull. 29:3363-3368, 1981; Wakiyama et al. “Preparation and evaluation in vitro and in vivo of polylactic acid microspheres containing dibucaine” Chem. Pharm. Bull. 30:3719-3727, 1982; each of which is incorporated herein by reference), in a blinded study. This comparison is particularly valuable since the biocompatibility and inflammatory potential of α-hydroxy acids such as PLGA (Cutright et al. “Histologic comparison of polylactic and polyglycolic acid sutures” Oral Surg. 32:165-173, 1971; Athanasiou et al. “Sterilization, toxicity, biocompatibility and clinical appilcations of polylactic acid/polyglycolic acid copolymers” Biomaterials 17:93-102, 1996; Brazeau et al. “Evaluation of PLGA microsphere size effect on myotoxicity using the isolated rodent skeletal muscle model” Pharm. Dev. Technol. 1:279-283, 1996; van der Elst et al. “Bone tissue response to biodegradable polymers used for intramedullary fracture fixation: A long-term in vivo study in sheep femora” Biomaterials 20: 121-128, 1999; each of which is incorporated herein by reference) when applied perineurally (Drager et al. “Prolonged intercostal nerve blockade in sheep using controlled release bupivacaine and dexamethasone from polyester microspheres” Anesthesiology 89: 969-979, 1998; incorporated herein by reference) has been described. As many parameters as possible were kept constant between the groups (means and site of drug administration, weight of particle delivered per rat, behavioral observer, dissection and histological techniques and time intervals), and observations were made in a blinded manner.

### Materials and Methods

Materials

Bupivacaine hydrochloride, human serum albumin (Fraction V), and lactose β-monohydrate were purchased from Sigma Chemical Co. (St. Louis, Mo.), L-α-dipalmitoylphosphatidylcholine (DPPC) from Avanti Polar Lipids (Alabaster, Ala.), poly (lactic-co-glycolic) acid (lactide:glycolide=65:35, MW 110,000) (PLGA₁₁₀) from Alkermes (Cambridge, Mass.), poly (lactic-co-glycolic) acid (lactide:glycolide=50:50, MW 20,000) (PLGA₂₀) from Boehringer Ingelheim (Ingelheim, Germany), poly (vinyl alcohol) (88% hydrolyzed, MW 20,000) from Polysciences (Warrington, Pa.), and USP grade ethanol from Pharmco Products (Brookfield, Conn.). Bupivacaine hydrochloride was made into the free base by alkaline precipitation and filtration.

Preparation of Spray-Dried Lipid-Protein Particles (LPSP) and PLGA Microspheres

LPSPs and PLGA microspheres were prepared and characterized (Kohane et al. “Sciatic nerve blockade with lipid-protein-sugar particles containing bupivacaine” Pharm. Res. 2000 (in press); incorporated herein by reference). In brief, LPSP were produced as follows. Dipalmitoylphosphatidyl-choline (DPPC) and bupivacaine free base were dissolved in ethanol, and albumin and lactose were dissolved in water. The two solution were mixed (so the final proportion (w/w) of solutes was DPPC 54: albumin 18: lactose 18: bupivacaine 10), and spray-dried using a Model 190 bench top spray drier (Büchi Co, Switzerland). PLGA microspheres containing 50% and 0% (w/w) bupivacaine were prepared by the single emulsion method using PLGA₁₁₀. Polymer and bupivacaine free base (200 mg total mass) were dissolved in 1.5 ml methylene chloride, and added to a solution of 1% polyvinyl alcohol in 100 mM Trizma buffer pH 8.5. The mixture was homogenized (Silverson L4R, Silverson Machines Ltd., Cheshire, England) at 3000 rpm, and methylene chloride was removed by rotary evaporation (Büchi Rotavap, Büchi, Switzerland) at 37° C. Spheres 20 μm to 120 μm in diameter were isolated by wet sieving (Newark Wire Co., Newark, N.J.), washed three times with water by centrifugation, then lyophilized to dryness. A separate group of 10% (w/w) bupivacaine microspheres were produced with PLGA₂₀. Twenty milligrams of bupivacaine and 180 mg of PLGA₂₀ were dissolved in 5 ml methylene chloride. The mixture was treated as above except that a) homogenization was performed at 9000 rpm, and b) following rotary evaporation the particles were collected in an Erlenmeyer flask (rather than in sieves). The purpose of these changes in method was to produce microspheres that were comparable to the LPSPs in size. Particle size was determined with a Coulter Multisizer (Coulter Electronics Ltd., Luton, U.K.).

Animal Care

Young adult male Sprague-Dawley rats weighing 310-420 g each were obtained from Taconic Farms (Germantown, N.Y.), and housed in groups in a 6 AM-6 PM light-dark cycle. Animals were cared for in compliance with protocols approved by the Animal Care and Use Committee at the Massachusetts Institute of Technology. NIH guidelines for the care and use of laboratory animals (NIH Publication #85-23 Rev. 1985) were observed. Rats were only injected once.

Perineural Injection Technique

Nerve block injections were performed via a 20 gauge needle under halothane-oxygen anesthesia as described (Kohane et al. “Sciatic nerve blockade with lipid-protein-sugar particles containing bupivacaine” Phann. Res. 2000 (in press); incorporated herein by reference). In brief, each rat was injected with a suspension of 75 mg of LPSPs or microspheres suspended in 0.6 ml of 1% sodium carboxymethyl cellulose, 0.1% Tween 80 (Castillo et al “Glucocorticoids prolong rat sciatic nerve blockade in vivo from bupivacaine microspheres” Anesthesiology 85:1157-66, 1996; Curley et al. “Prolonged regional nerve blockade. Injectable biodegradable bupivacaine/polyester microspheres” Anesthesiology 84:1401-1410, 1996; Drager et al. “Prolonged intercostal nerve blockade in sheep using controlled release bupivacaine and dexamethasone from polyester microspheres” Anesthesiology 89: 969-979, 1998; each of which is incorporated herein by reference) after gentle agitation (<5 sec) in preparation for injection. The presence of nerve block (and therefore proper location of the injected particles) was confirmed by hotplate testing (Kohane et al. “Sciatic nerve blockade with lipid-protein-sugar particles containing bupivacaine” Phann. Res. 2000 (in press); incorporated herein by reference) in all animals, except those injected with blank (no bupivacaine) particles.

Scoring of Autotomy and Touch-Evoked Agitation

Each rat was scored for daily autotomy (Wall et al. “Autotomy following peripheral nerve lesions: experimental anaesthesia dolorosa” Pain 7:103-111, 1979; incorporated herein by reference) as follows. One point was given for mutilation of one or more nails on the hindpaw. An additional point was assigned for each distal (up to 5 points) and proximal (up to 5 points) half-digit attacked by the rat. Thus the score could range from 0 to 11. Rats who achieved a score of 11 were sacrificed.

The site of injection and ipsilateral leg of each rat was lightly palpated for touch-evoked agitation at hourly intervals for the first eight hours, then daily, using the following scoring system (Yanez et al. “Touch-evoked agitation produced by spinally administered phospholipid emulsion and liposomes in rats” Anesthesiology 82:1189-1198, 1995). Normal behavior to touch =0; occasional squeaking, mild agitation to touch=1; spontaneous agitation or squeaking or severe agitation to touch=2.

Rat Sciatic Nerve Dissection

Under deep halothane/oxygen general anesthesia, the sciatic nerve was exposed by a longitudinal incision on the lateral thigh, followed by careful separation of the muscles of the anterior and posterior thigh. The wound was extended proximally until the entire area of injection (the area posteromedial to the greater trochanter) was fully revealed. The nerve was then cut above the greater trochanter and at the trifurcation and placed into 4% formaldehyde at 4° C. Intraperitoneal pentobarbital (100 mg/kg) was administered following removal of the nerve.

The dissector was blinded as to which type of particle each rat had been injected with. At the time of dissection, the degree to which the tissues surrounding the nerve were matted together was scored as follows: “0”: tissue planes obvious and easily separated, “1”: tissue planes obvious but separated with some difficulty, “2”: tissue planes effaced and separated with some difficulty, “3”: tissue planes completely obliterated, could not separate surrounding tissues from nerve without cutting through them.

Histological Preparations

Embedding, sectioning, and staining with hematoxylin/eosin of dissected nerves were performed using standard techniques. The amount of inflammation was estimated by cell counting in the most inflamed areas of the sections (selected under light microscopy). The observer was blinded as to the type of particles present in each sample. Cell counts were performed on digitized images obtained using light microscopy at 400× magnification. Image size was calculated using a calibration micrometer. The area of inflammation in each dissected nerve was estimated by measuring the long and short axes of the mass and assuming a generally rectangular shape.

Statistical Analysis

Neurobehavioral data are reported as means with standard deviations. Comparisons between groups of such data were made using Student's t-test. Non-parametric data (dissection scores, presence vs. absence of polymer residue) and data that were not normally distributed (area of inflammation) were compared using the Mann-Whitney U-test.

### Results

Characteristics of LPSPs and PLGA Microspheres

The data relating to the production and characterization of the LPSPs and PLGA₁₁₀ microspheres has been described in Example 1 and elsewhere (Kohane et al. “Sciatic nerve blockade with lipid-protein-sugar particles containing bupivacaine” Pharm. Res. 2000 (in press); incorporated herein by reference). Relevant aspects are summarized in Table 2, together with data on PLGA₂₀ microspheres.

TABLE 2
Characteristics of particles

| | Composition | | |
|---|---|---|---|
| | | Bupivacaine¹ | Median particle |
| Particle Type | Polymer | % (w/w) | diameter (μm) |
| LPSP² | —³ | 10% | 4.4 ± 0.4 |
| PLGA⁴ | PLGA₁₁₀ | 50% | 59 ± 12 |
| | PLGA₂₀ | 10% | 3.6 ± 0.2 |

¹Theoretical loading. Actual loading was approximately 80% of this value (Kohane et al. “Sciatic nerve blockade with lipid-protein-sugar particles containing bupivacaine” Pharm. Res. 2000 (in press); incorporated herein by reference).
²Lipid proteins sugar particles.
³The excipients are dipalmitoylphosphatidylcholine, albumin, and lactose.
⁴Poly (lactic-co-glycolic) acid. The subscript refers to the molecular weight. See Materials for further details.

Tissue Reaction

Groups of rats were injected at the sciatic nerve with 10% (w/w) bupivacaine LPSPs or 50% (w/w) PLGA₁₁₀ microspheres. The sciatic nerves were removed 4 days (n=4), 2 weeks (n =6), or 7 months (n=4, LPSP; n=5, PLGA₁₁₀) after injection and processed for histology, together with adherent tissues.

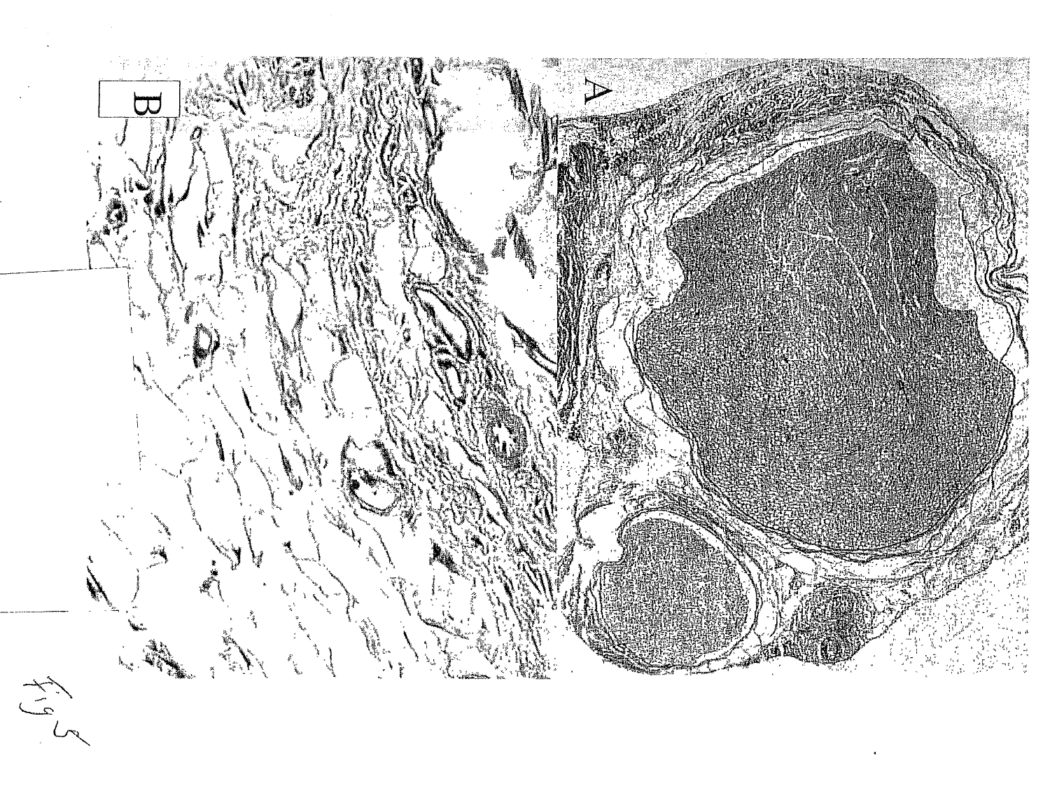

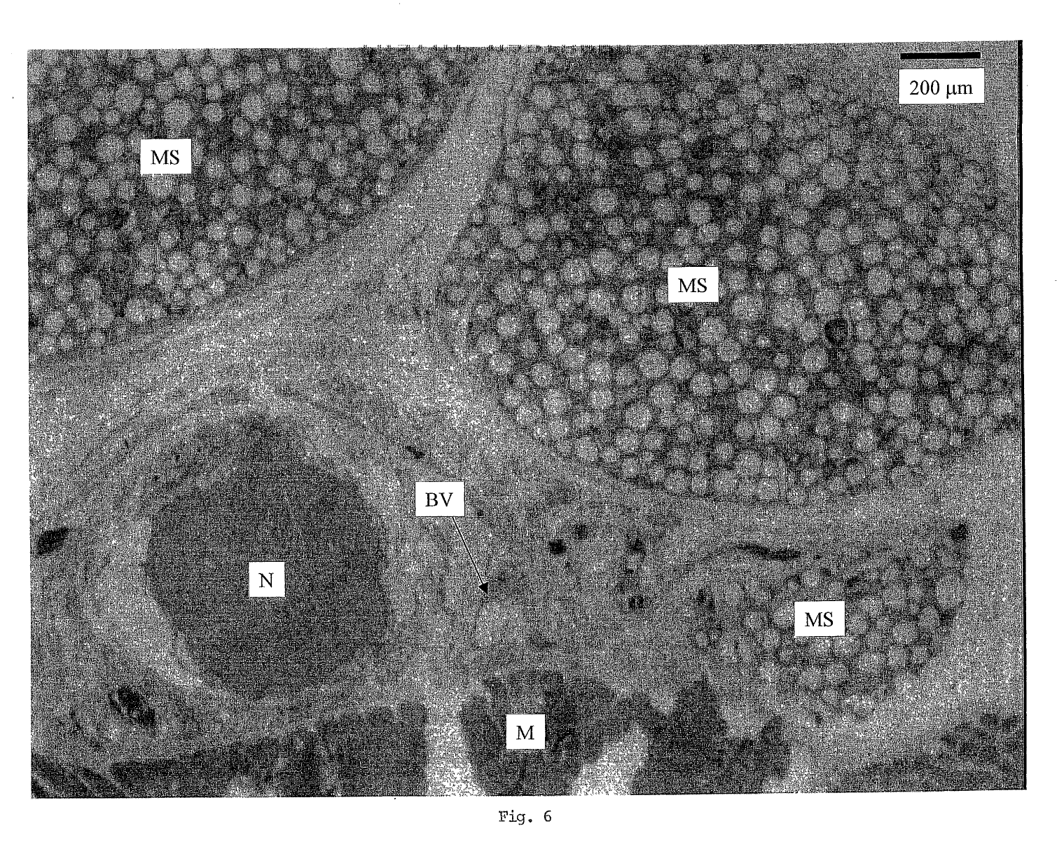

General observations. FIG. 5 shows the histological appearance of a control nerve sciatic nerve. FIG. 6 is a representative photomicrograph demonstrating the delivery of particles (in this case PLGA₁₁₀ microspheres) to the vicinity of the sciatic nerve. If detectable, both particle types were localized in this manner at all time points (except as described below). In general (and at all time points examined), the inflammatory response to PLGA₁₁₀ microspheres was confined to clearly demarcated pockets. Inflammation from LPSP was found more diffusely throughout the tissues. In general, neither type of LPSPs nor PLGA₁₁₀ microspheres were found within the perineurium. There was no clinical or histological evidence of infection in any of the animals at any time point.

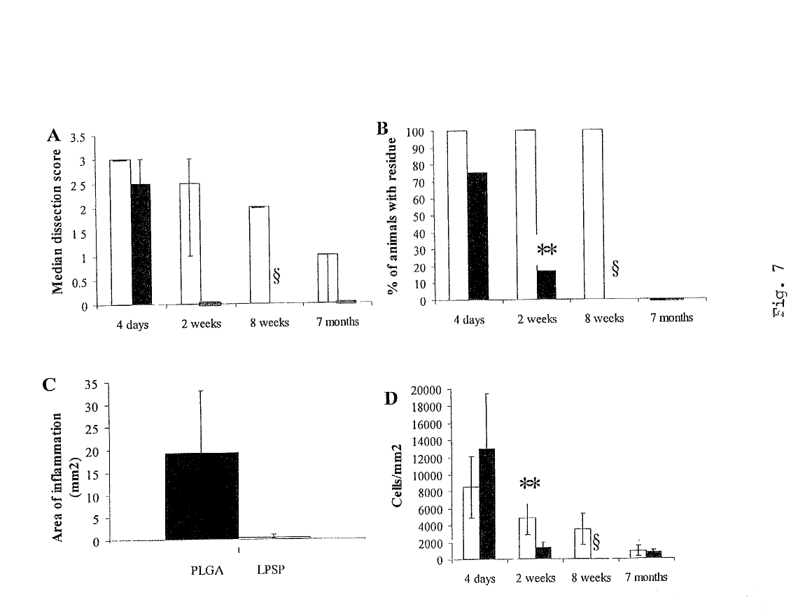

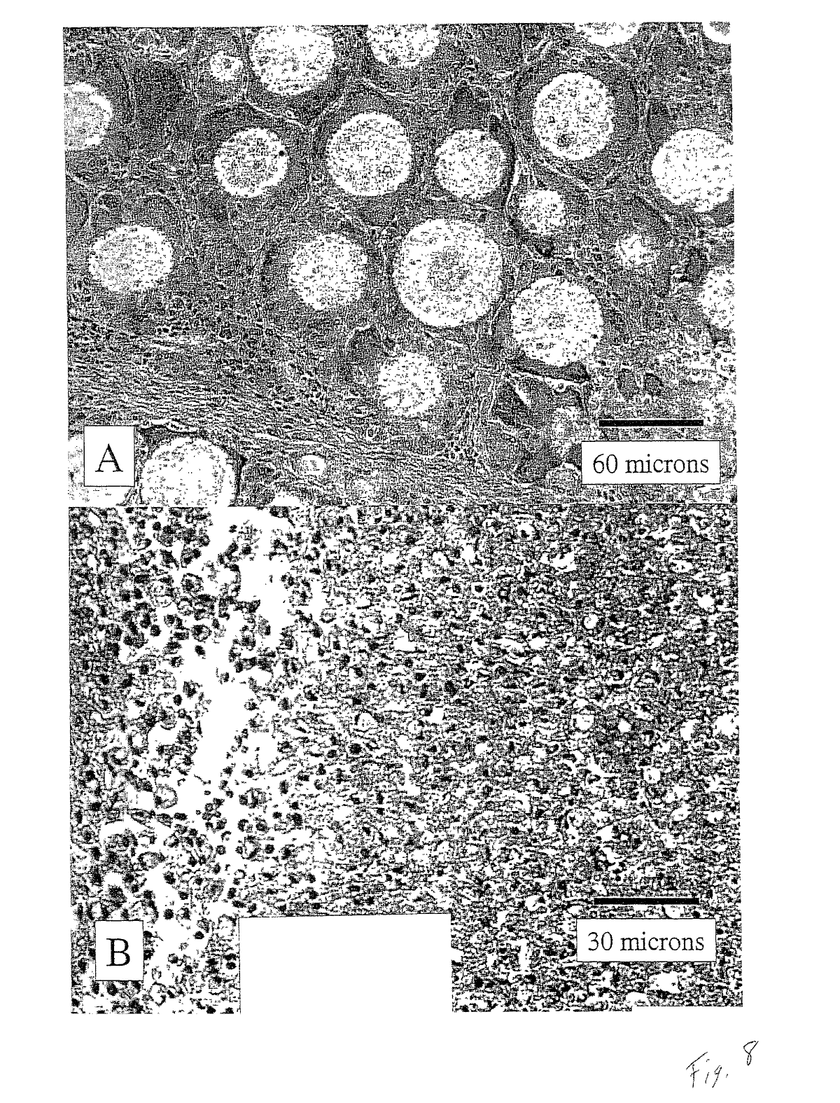

Tissue reaction four days after injection. All rats injected with PLGA₁₁₀ microspheres had large, firm, slightly gritty deposits of particles in discrete globules around the nerve. Three out of four rats that received LPSP had visible deposits of particles, which were much smaller than the PLGA₁₁₀ deposits and were soft to the touch. There was no statistically significant difference between the PLGA₁₁₀ and LPSP groups in dissection scores, presence of polymer residue, or cell density (FIG. 7). The area of inflammation was not quantitated because it was too large and amorphous in the PLGA group to be dissected en bloc. Both groups had large areas of inflammation and high cell densities on light microscopy, there were marked histological differences. As demonstrated in FIG. 8, the PLGA₁₁₀ group showed a foreign-body-type granulomatous reaction with multinucleated giant cells surrounding the microspheres (which appear as empty circles 50 to 90 μm across, some with traces of polymer remaining). The LPSP group showed mostly acute inflammation with lymphocytes and macrophages and occasional neutrophils and foamy macrophages. LPSP were not discernible as discrete particles, but as an area of diffuse pink staining. In some animals in both groups, inflammation also involved adjacent muscle and perineural adipose tissue, with necrotic muscle fibers, myophagocytosis, and myocyte regeneration-all signs of acute muscle injury. Necrosis was associated with acute inflammation, with neutrophils forming small sterile microabscesses.

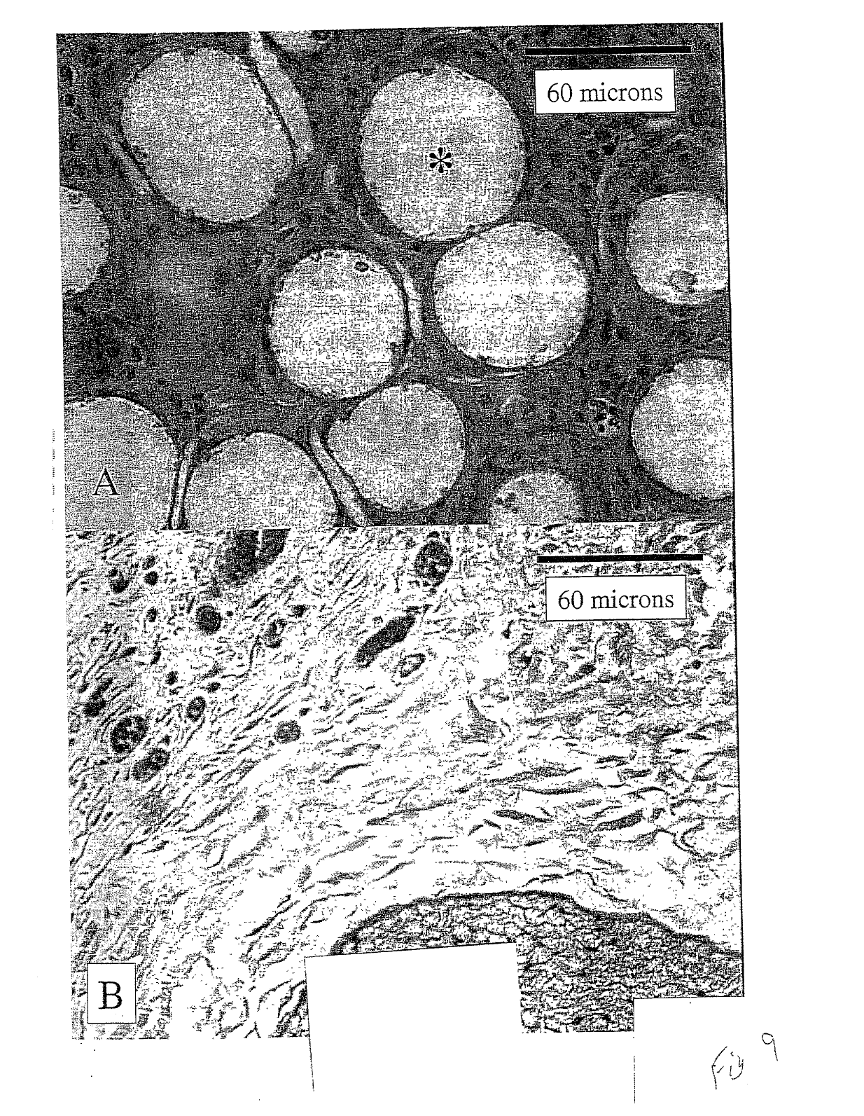

Tissue reaction two weeks after injection. Deposits of microspheres were still found in all rats injected with PLGA₁₁₀ microspheres; these were often several millimeters across. In comparison, a very small amount of residue (approximately 0.5 mm by 3 mm) was found in only one of the rats injected with LPSPs. This difference in incidence was statistically significant (p=0.005). PLGA₁₁₀ microspheres were surrounded with a large dense granulomatous foreign body reaction (FIG. 9a) with lymphocytes and macrophages, while LPSPs had a small loose, predominantly lymphocytic infiltrate (FIG. 9b).

Microscopic examination of histological preparations from rats injected with PLGA₁₁₀ microspheres revealed 4800±1900 cells/mm², which was 3.4 times more (p=0.006) than the 1400±600 cells/mm² seen in those injected with LPSP (FIG. 7). The area of inflammation was also much larger in the PLGA₁₁₀ group (FIG. 7, p=0.01). One PLGA₁₁₀-treated nerve showed neutrophils and active myositis. In another PLGA₁₁₀-treated rat, a massive cavitary mass was found at the injection site, with a pronounced granulomatous reaction and a large degree of axonal degeneration, on the side of the nerve facing the granuloma.

Tissue reaction eight weeks after injection. An additional group who received PLGA₁₁₀ microspheres was harvested eight weeks after injection (n=5). All five rats showed large amounts of polymer residue. All rats had a dissection score of 2. The histological appearance was comparable to that seen at 2 weeks, with a cell density of 3500±1800 cells/mm². There was no counterpart group for LPSPs because there was already almost no particle mass to follow at two weeks. (Note the very small size of the inflammatory masses in the LPSP group at 2 weeks, FIG. 7c). The cell count and incidence of residue in the PLGA₁₁₀ group was higher at 8 weeks than the corresponding values had been in the LPSP group at 2 weeks (six weeks earlier).

Tissue reaction seven months (210 days) after injection. All of the dissections of LPSP-injected rats (n=4) were scored as “0”. In the PLGA₁₁₀ group (n=5), two had a score of “1”, and one had a score of “2” (p=0.08). No pockets of polymer residue were visible at dissection in either group. Microscopic examination of most samples from both groups at this time point were felt to be either entirely normal, or to have slightly increased cellularity with a loose architecture, suggesting the possibility of edema, with several lymphocytes. One sample from the PLGA₁₁₀ groups showed a small foreign body reaction around a piece of extraneous material. There was no statistically significant difference in cell density between groups, nor did either represent a significant increase over the cellularity of control nerves (370±40 cells/mm², p=n.s. vs. both particle groups). There was no obvious area of inflammation to measure in either group.

In order to assess the contribution of particle size to the differences noted between LPSPs and PLGA microspheres, four rats were injected with PLGA₂₀ microspheres 3.6±0.2 μm in diameter (vs. 4.4±0.4 μm for the LPSPs) loaded 10% (w/w) with bupivacaine. (In order to further minimize the dwell time of the microspheres we used PLGA₂₀, a polymer that has a much lower molecular weight and higher proportion of glycolic to lactic acid monomers than PLGA₁₁₀). Two weeks after injection, the median dissection score was 3, all four rats had large pockets of polymer residue, and the cell density was 7356±1604 cells/mm² (p=0.06 compared to PLGA₁₁₀ group). The outlines of microspheres were still visible on histology. There were many macrophages and lymphocytes, and occasional giant cells (a much lower incidence of the latter than with the PLGA₁₁₀ microspheres).

Other Findings on Dissection

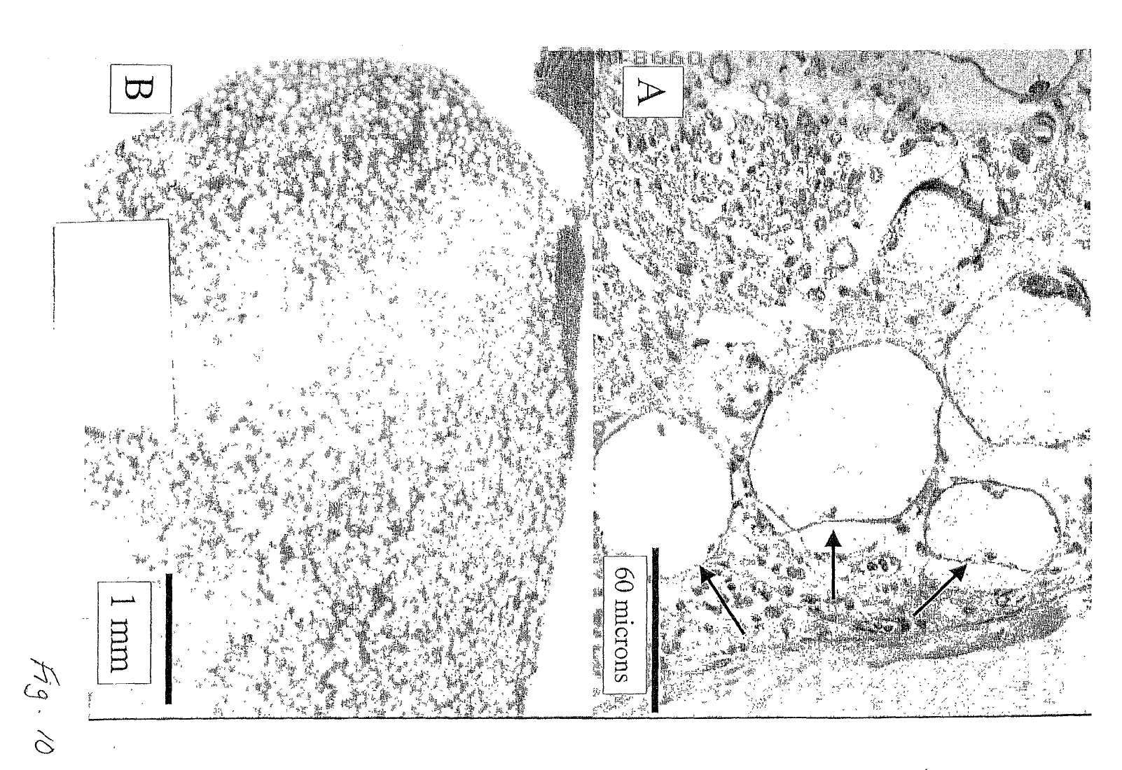

In some animals injected with PLGA₁₁₀ microspheres, particles were found at locations where they were not intentionally placed. In the group dissected four days after injection, one of the animals had a continuous cord of particles that extended almost to the knee from the site of injection, and two others had visible particle residue tracking up into the subcutaneous tissue. Similar findings occurred in the group dissected two weeks after injection. In one rat, microspheres were found within the nerve, beneath the perineural sheath (FIG. 10a), with infiltration of inflammatory cells. In another rat, a mass of microspheres surrounded by inflammation, measuring 8 mm×0.4 mm×2 mm was found 2.5 cm distal to the site of injection along the course of the sciatic nerve, near the knee (FIG. 10b). There were no similar findings in rats injected with LPSPs.

Touch-Evoked Agitation, Distress, and Autotomy Scoring

All rats (PLGA₁₁₀-, PLGA₂₀-, and LPSP-treated groups) were palpated at the site of injection and on the ipsilateral leg as per Methods. None of the rats in any of the groups appeared distressed by this maneuver (score=0). Furthermore, no rat had an autotomy score above zero. All the rats appeared well groomed, and continued to gain weight throughout the duration of the experiment.

All of the rats in this study had full recovery of sensory and motor function in the injected extremity when the local anesthetic effect of the microparticles resolved (data not shown), and none had any detectable signs of long-term functional deficits or pain-related behavior.

### Discussion

The primary focus of this study was to compare the tissue reactions to LPSPs and PLGA microspheres that had previously been shown to cause approximately equivalent durations of sciatic sensory nerve blockade in the rat (Kohane et al. “Sciatic nerve blockade with lipid-protein-sugar particles containing bupivacaine” Pharm. Res. 2000 (in press); incorporated herein by reference).

There were striking differences in the degree of inflammatory response to the LPSP and PLGA particles at two weeks after injection. There was only a mild patchy lymphocytic infiltrate around nerves where LPSPs had been injected. In contrast, the PLGA₁₁₀ microspheres produced a tissue response that was consistent with the observations of other investigators (Drager et al. “Prolonged intercostal nerve blockade in sheep using controlled release bupivacaine and dexamethasone from polyester microspheres” Anesthesiology 89: 969-979, 1998; van der Elst et al. “Bone tissue response to biodegradable polymers used for intramedullary fracture fixation: A long-term in vivo study in sheep femora” Biomaterials 20: 121-128, 1999; each of which is incorporated herein by reference), with pronounced inflammation at least as far out as 8 weeks (n =5, data not shown). Traces of the reaction to PLGA₁₁₀ were seen as far out as 7 months after injection. Since particle residue was noted for a much longer time in rats injected with PLGA microspheres than LPSPs, and the durations of anesthetic effect were similar, the ratio of duration of therapeutic effect to duration of polymer residue is better for the LPSPs. This suggests that the latter may be more suitable for repeated injections, particularly at the same site.

Although the long-term biocompatibility of the LPSPs was superior to that of the PLGA microspheres, this may not be related to the fact that the excipients were naturally occurring in the human body. This is shown by the fact that there was marked inflammation at 4 days post-injection. Instead, the improved long-term biocompatibility may be due to the much shorter dwell time of the particles in the tissue.

The shorter duration of inflammation from LPSPs could be due to their being much smaller than the PLGA₁₁₀ microspheres and therefore more easily taken up and removed by leukocytes (Tabata et al. “Phagocytosis of polymer microspheres by macrophages” Adv. Polymer Sci. 94:107-141; 1990; incorporated herein by reference). The fact that 3.6 μm diameter PLGA₂₀ microspheres also showed prolonged inflammation argues against a size difference being the sole explanation. The differences in duration of inflammatory response may therefore be partly due to the materials used per se. (Note that we used a relatively rapidly degrading PLGA in the smaller microspheres; nevertheless particles were still visible on histology).

The nature of the tissue response elicited by the LPSPs (acute inflammation) at all time points was different from that seen with PLGA₁₁₀ microspheres (granulomatous foreign body reaction). It is possible that this difference is due to the difference in particle size, as suggested by the fact that the 3.6 μm PLGA₂₀ microspheres showed less of a giant cell foreign body type reaction than was seen with the larger microspheres. This is consistent with fact that macrophages tend to ingest small particles, but tend to form multinucleated giant cells to surround objects larger than themselves.

It bears mentioning that the tissue reaction to both particle types was not due to the encapsulated bupivacaine. Blank (no drug) LPSPs and PLGA₁₁₀ microspheres (n=4 each) produced the same qualitative and quantitative tissue effects seen with drug-loaded particles two weeks after injection.

Any material that is injected blindly (i.e., not under direct visualization) into the body has the potential for being injected at a site other than the intended target, or of being injected at the correct location but being pushed away by the force of injection, or of migrating even if deposited at the correct location. These possibilities were demonstrated in the rat in which inadvertent intraneural injection occurred, and in the ones in which large inflammatory masses were found tracking to the knee. Those cases point out the potential hazards when a) particles are injected either near a vital or sensitive structure or b) the site of injection itself does not confine the particles to a locale effectively. Although it is probably equally likely for such events to occur with either type of particle, particles which disappear more rapidly from the tissues and that cause less long-term inflammation may be safer. Given the potentially severe sequelae of having an inflammatory mass extending into a nerve, it will probably be advisable to take special precautions (nerve stimulator, radiological guidance) when performing blocks with such particles at sites where a major nerve could be hit.

None of the rats injected with either type of particle showed any neurological deficits, even where there was marked inflammation or intraneural microspheres. The mean thermal latency prior to sacrifice was comparable to that in pristine rats. None of the rats showed any “touch-evoked agitation” or autotomy.

### Example 3

Preparation of LPSPs Containing Nucleic Acids

The following Examples describes the encapsulation of DNA in a lipid-protein-sugar microparticle. The DNA encapsulated in the microparticles was then isolated from the LPSPs to demonstrate that the DNA is largely undamaged by the encapsulation procedure.

Dipalmitoylphosphatidylcholine (300 mg) was dissolved in ethanol, and lactose (100 mg), albumin (100 mg), and DNA (1 mg) containing the gene encoding•-galactosidase were dissolved in distilled water (37.5 mL). The two solutions were mixed, and the resulting mixture was spray dried using a Buchi 190 bench-top spray drier. The resulting microparticles containing DNA were then analyzed to determine the affect of the encapsulation procedure on the nucleic acid.

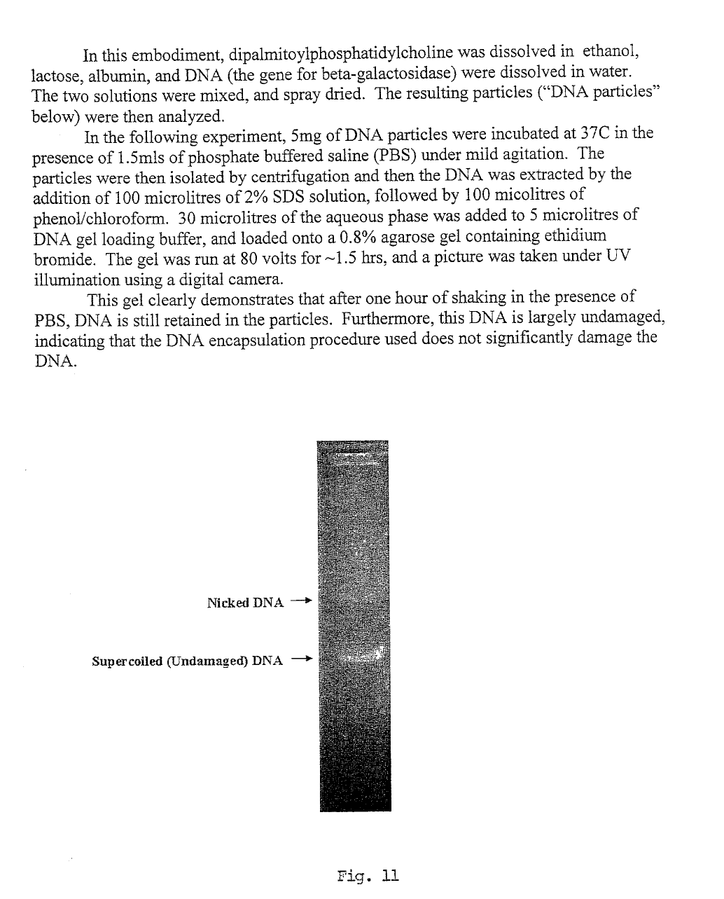

Microparticles containing DNA (5 mg) were incubated at 37° C. in the presence of phosphate buffer saline (PBS) solution (1.5 mL) under mild agitation. The microparticles were then isolated by centrifugation, and then the DNA was extracted by the addition of 2% SDS solution (100•L), followed by phenol/chloroform (100•L). The aqueous phase (30•L) was added to DNA gel loading buffer (5•L), and the resulting solution was loaded onto a 0.8% agarose gel containing ethidium bromide. The gel was run at 80 V for ˜1.5 hours, and a picture of the gel (FIG. 11) was taken under UV illumination using a digital camera.

The gel clearly demonstrated that after one hour of shaking in the presence of PBS solution, the DNA is still retained in the particles. Furthermore, the DNA extracted from the LPSPs is largely undamaged, indicating that the encapsulation procedure does not significantly damage the DNA.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A pharmaceutical composition comprising microparticles of a polynucleotide encapsulated in a matrix comprising lipid, protein, and sugar.

2. A pharmaceutical composition comprising microparticles of a polynucleotide encapsulated in a matrix, wherein the matrix comprises at least three components selected from the group consisting of lipid, protein, sugar, and synthetic polymer.

3. A pharmaceutical composition comprising microparticles of a polynucleotide encapsulated in a matrix, wherein the matrix comprises at least two components selected from the group consisting of lipid, protein, sugar, and synthetic polymer.

4. A pharmaceutical composition comprising microparticles of a polynucleotide encapsulated in a matrix comprising lipid and protein.

5. A pharmaceutical composition comprising microparticles of a polynucleotide encapsulated in a matrix comprising lipid and sugar.

6. A pharmaceutical composition comprising microparticles of a polynucleotide encapsulated in a matrix comprising protein and sugar.

7. The pharmaceutical composition of claim 1 wherein the polynucleotide is DNA.

8. The pharmaceutical composition of claim 1 wherein the polynucleotide is RNA.

9. The pharmaceutical composition of claim 1 wherein the polynucleotide comprises RNA and DNA.

10. The pharmaceutical composition of claim 1 wherein the polynucleotide is a modified polynucleotide.

11. The pharmaceutical composition of claim 1 wherein the polynucleotide is a derivative of DNA or RNA.

12. The pharmaceutical composition of claim 1 wherein the polynucleotide is at least 100 base pairs in length.

13. The pharmaceutical composition of claim 1 wherein the polynucleotide is at least 1000 base pairs in length.

14. The pharmaceutical composition of claim 1 wherein the polynucleotide is at least 10000 base pairs in length.

15. The pharmaceutical composition of claim 1 wherein the polynucleotide is a plasmid.

16. The pharmaceutical composition of claim 1 wherein the polynucleotide encodes a pharmaceutically active protein.

17. The pharmaceutical composition of claim 1 wherein the polynucleotide encodes an immunologically active protein.

18. The pharmaceutical composition of claim 1 wherein the polynucleotide encodes a bacterial protein antigen.

19. The pharmaceutical composition of claim 1 wherein the polynucleotide encodes a viral protein antigen.

20. The pharmaceutical composition of claim 1 wherein the lipid is a naturally occurring lipid.

21. The pharmaceutical composition of claim 1 wherein the lipid is an emulsifier.

22. The pharmaceutical composition of claim 1 wherein the lipid is a surfactant.

23. The pharmaceutical composition of claim 1 wherein the lipid is positively charged.

24. The pharmaceutical composition of claim 1 wherein the lipid is negatively charged.

25. The pharmaceutical composition of claim 1 wherein the lipid has no charge.

26. The pharmaceutical composition of claim 1 wherein the lipid is a phosphatidylcholine.

27. The pharmaceutical composition of claim 1 wherein the lipid is dipalmitoylphosphatidylcholine (DPPC).

28. The pharmaceutical composition of claim 1 wherein the lipid is polyvinyl alcohol.

29. The pharmaceutical composition of claim 1 wherein the lipid is a phospholipid.

30. The pharmaceutical composition of claim 1 wherein the lipid is selected from the groups consisting of phosphoglycerides; phosphatidylcholines; dipalmitoyl phosphatidylcholine (DPPC); dioleylphosphatidyl ethanolamine (DOPE); dioleyloxypropyltriethylamrnmonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid amides; sorbitan trioleate (Span 85) glycocholate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-5 amine; acetyl palmitate; glycerol ricinoleate; hexadecyl sterate; isopropyl myristate; tyloxapol; poly(ethylene glycol)5000-phosphatidylethanolamine; and phospholipids.

31. The pharmaceutical composition of claim 1 wherein the lipid is a derivatized lipid.

32. The pharmaceutical composition of claim 1 wherein the protein is an albumin.

33. The pharmaceutical composition of claim 1 wherein the protein is a whole cell extract.

34. The pharmaceutical composition of claim 1 wherein the protein is an antibody.

35. The pharmaceutical composition of claim 1 wherein the protein is an enzyme.

36. The pharmaceutical composition of claim 1 wherein the protein is glucose oxidase.

37. The pharmaceutical composition of claim 1 wherein the sugar comprises a mixture of complex and simple sugars.

38. The pharmaceutical composition of claim 1 wherein the sugar is lactose.

39. The pharmaceutical composition of claim 1 wherein the sugar is cellulose.

40. The pharmaceutical composition of claim 1 wherein the sugar is a chemically modified sugar.

41. The pharmaceutical composition of claim 1 wherein the sugar is a glycosaminoglycan.

42. The pharmaceutical composition of claim 1 wherein the sugar is dextran.

43. The pharmaceutical composition of claim 1 wherein the sugar is chemically modified dextran.

44. The pharmaceutical composition of claim 1 wherein the sugar is chondroitin sulfate.

45. The pharmaceutical composition of claim 1 wherein the sugar is a derivatized sugar.

46. The pharmaceutical composition of claim 1 wherein the sugar is a chemically modified sugar.

47. The pharmaceutical compostion of claim 1 wherein the sugar is selected from the group consisting of galactose, lactose, glucose, maltose, starches, cellulose and its derivatives, methyl cellulose, carboxymethyl cellulose, fructose, dextran and its derivatives, raffinose, mannitol, xylose, dextrins, glycosaminoglycans, sialic acid, chitosan, hyaluronic acid, and chondroitin sulfate.

48. The pharmaceutical composition of claim 1 wherein the ratio of lipid to protein to sugar is approximately 3:1:1.

49. The pharmaceutical composition of claim 1 wherein the lipid comprises 0-99% of the matrix by weight.

50. The pharmaceutical composition of claim 1 wherein the lipid comprises 3-99% of the matrix by weight.

51. The pharmaceutical composition of claim 1 wherein the lipid comprises 20-60% of the matrix by weight.

52. The pharmaceutical composition of claim 1 wherein the protein comprises 0-95% of the matrix by weight.

53. The pharmaceutical composition of claim 1 wherein the protein comprises 10-30% of the matrix by weight.

54. The pharmaceutical composition of claim 1 wherein the protein comprises 1-20% of the matrix by weight.

55. The pharmaceutical composition of claim 1 wherein the sugar comprises 0-60% of the matrix by weight.

56. The pharmaceutical composition of claim 1 wherein the sugar comprises 0.5%-50% of the matrix by weight.

57. The pharmaceutical composition of claim 1 wherein the sugar comprises 10-30% of the matrix by weight.

58. The pharmaceutical composition of claim 1 wherein the microparticles are less than 50 micrometers in diameter.

59. The pharmaceutical composition of claim 1 wherein the microparticles are less than 10 micrometers in diameter.

60. The pharmaceutical composition of claim 1 wherein the microparticles are less than 5 micrometers in diameter.

61. The pharmaceutical composition of claim 1 wherein the microparticles are less than 1 micrometer in diameter.

62. The pharmaceutical composition of claim 1 wherein the microparticles are less than 500 nanometers in diameter.

63. A method of preparing microparticles comprising a polynucleotide encapsulated in a lipid-protein-sugar matrix, the method comprising steps of:
providing a polynucleotide;
contacting the polynucleotide with a lipid, a protein, and a sugar; and
spray drying mixture of the polynucleotide, the lipid, the protein, and the sugar to make microparticles.

64. A method of preparing microparticles comprising a polynculeotide encapsulated in a matrix, the method comprising steps of:
providing a polynucleotide;
contacting the polynucleotide with at least two components selected from the group consisting of a lipid, a protein, a sugar, and a synthetic polymer; and
spray drying polynucleotide and components to make microparticles.

65. A method of administering an agent, the method comprising steps of:
providing a patient;
providing microparticles of a polynucleotide encapsulated in a lipid-protein-sugar matrix; and
administering the microparticles to the patient.

66. The method of claim 65 wherein the step of administering comprises injecting the microparticles into the patient.

67. The method of claim 65 wherein the step of administering comprises placing the microparticles in a body cavity of the patient.

68. The method of claim 65 wherein the step of administering comprises inhaling the microparticles.

69. A method of transfecting cells, the method comprising steps of:
providing at least one cell;
providing lipid-protein-sugar particles containing a polynucleotide; and
contacting the cell with the particles.

70. The method of claim 69 wherein the cells are in vitro.

71. The method of claim 69 wherein the cells are hematopoietic stem cells.

72. The method of claim 69 wherein the cells are embryonic stem cells.

73. A method of immunizing an individual, the method comprising steps of:
providing an individual;
providing microparticles comprising a polynucleotide encapsulated in a lipid-protein-sugar matrix; and
delivering an effective amount of the microparticles to the individual to stimulate an immune response.

74. The method of claim 73 wherein the polynucleotide encodes a protein antigen.

75. The method of claim 74 wherein the protein antigen is derived from bacteria, viruses, protozoa, or parasites.

76. The method of claim 73 wherein the microparticles further comprise an adjuvant.

77. The method of claim 73 wherein the microparticles are less than 5 micrometers in diameter.

78. The method of claim 73 wherein the microparticles are at least 5 micrometers in diameter.
